(19) United States
(12) Patent Application Publication
(10) Pub. No.: US 2023/0066280 A1
(43) Pub. Date: Mar. 2, 2023

(54) DRUG ELUTING BIOMATERIALS

(71) Applicant: UCL BUSINESS LTD, London (GB)

(72) Inventors: Melissa RAYNER, London (GB); James PHILLIPS, London (GB); Jess HEALY, London (GB); Duncan CRAIG, London (GB); Gareth WILLIAMS, London (GB); Ana Rita Marques Pereira TRINDADE, London (GB); Karolina DZIEMIDOWICZ, London (GB); Victoria ROBERTON, London (GB); Holly GREGORY, London (GB); Desponia ELEFTHERIADOU, London (GB)

(73) Assignee: UCL BUSINESS LTD, London (GB)

(21) Appl. No.: 17/793,221

(22) PCT Filed: Jan. 18, 2021
(86) PCT No.: PCT/GB2021/050108

§ 371 Date: Jul. 15, 2022

(30) Foreign Application Priority Data
Jan. 17, 2020 (GB) 2000714.2

(51) Int. Cl. A61L 27/36; A61L 27/18; A61L 27/54; A61L 27/58; A61K 31/192

(52) U.S. Cl. CPC A61L 27/3675; A61L 27/18; A61L 27/54; A61L 27/58; A61K 31/192; A61L 2400/12

(57) ABSTRACT

The invention relates to a nanofibrous material comprising a drug for treating a peripheral nerve injury by delivering the drug locally to a damaged or injured nerve. The drug may be such a Non Steroidal Anti Inflammatory Drug or a PPAR agonist. In particular, the invention relates to a drug eluting nerve wrap or bandage that can be wrapped around an injured peripheral nerve. The invention also relates to a nanofibrous drug delivery system or device for delivering a drug locally to a peripheral nerve, a treatment for a peripheral nerve injury comprising contacting a damaged nerve with the drug eluting nanofibrous material or drug delivery system, kits and methods for making the nanofibrous materials, and uses of the nanofibrous materials.

### FIELD OF THE INVENTION

This invention relates to biomaterials, drug delivery systems, methods, uses, agents and compositions for treating damaged or injured nerves. In particular, the invention relates to drug-eluting materials that can be wrapped around a damaged nerve.

### BACKGROUND OF THE INVENTION

Peripheral nerve injury (PNI) incidence is 2-5% of trauma cases, affecting ˜1 million people in Europe and US p.a. of whom 600,000 have surgery, but only 50% regain function.

Poor clinical outcomes following PNI are partly attributable to the limited rate of neuronal regeneration. The neurons in the upstream (proximal) part of the nerve can regenerate and, after any gaps have been bridged, they will grow slowly all the way back to their targets (e.g. muscle and skin) to restore function. While advances in microsurgical techniques and nerve grafting have helped overcome problems associated with regeneration across gaps, there is still a fundamental challenge associated with the slow rate of neuron regeneration that limits restoration of function. This is because even in the best-case scenario of a successful repair procedure resulting in neurons growing in a supportive environment, regeneration is delayed and growth rate is limited (to approximately 1 mm per day). Consequently, especially with more proximal injuries, a long period of time (months to years) can elapse before the regenerating neurons reach their target organs. In the case of motor nerves growing back to muscles, this delay can be devastating, since muscles without nerve stimulation atrophy over time, wasting away irreversibly in many cases, so that even if neurons do successfully regenerate to their target muscle there is little recovery of function.

Despite numerous potential drug candidates demonstrating positive effects on nerve regeneration rate in preclinical models, no drugs are routinely used to improve restoration of function in clinical practice. A key challenge associated with clinical adoption of drug treatments in nerve injured patients is the requirement for sustained administration of doses associated with undesirable systemic side-effects.

PNI is associated with substantial socioeconomic impact as the resulting disability can be debilitating, significantly affecting the patient's quality of life^{1,2}. Treatments mainly employ microsurgical interventions although additional therapeutics that can be administered following PNI have emerged such as cell therapies, proteins, platelet-rich plasma and gene therapy³⁻⁶. There are currently no drug therapies that are routinely used to promote regeneration in nerve injured patients, despite various candidate drugs showing benefit in preclinical models⁷. One drug which has been shown to improve regeneration when administered systemically to rats following PNI is ibuprofen, a non-steroidal anti-inflammatory drug (NSAID) which is likely to act to accelerate neurite elongation as an agonist of peroxisome proliferator-activated receptor gamma (PPARγ)^{8, 9} Another NSAID with PPARγ agonist activity is sulindac sulfide, which has not previously been tested for use in nerve repair.

Pharmaceutical challenges that have prevented translation of drug therapies such as ibuprofen from preclinical to human usage in PNI include the provision of adequate drug dose and duration while reducing unwanted side effects associated with systemic administration. Long term oral treatment in particular can lead to side effects and also problems with patient compliance. For example, despite a generally acceptable safety profile, there are risks associated with systemic administration of PPAR agonists and NSAIDs continuously at high doses over a period of many weeks (Wright, M. B., et al., Minireview: Challenges and opportunities in development of PPAR agonists. Mol Endocrinol, 2014. 28(11): p. 1756-68).

WO-A-2019/239436 discloses electrospun fibres for local release of an anti-inflammatory agent and a promyelinating agent to limit secondary neurodegeneration triggered by glutamate release and supported by on-going inflammation in the nervous system, for the treatment of a spinal cord injury (SCI). US-A-2019/0083415 describes a sustained-release sheet that includes a drug, for treating a peripheral nerve injury. CN 102525689 describes an electrospun nerve repair conduit comprising Nerve Growth Factor to promote nerve regeneration. The conduit is used to bridge a gap in a transected peripheral nerve and guides the growth of nerve cells along the axis of the conduit. A tissue engineered nerve conduit is also described in US-A-2014/227339, which describes a scaffold comprising mesenchymal progenitor cells to improve wound healing, including to bridge gaps in transected nerves. CN 1061913393 also describes a nerve scaffold (conduit) for inducing nerve regeneration for gap repair of a transected nerve. A conduit for supporting nerve regeneration is also described in WO-A-2016/192733, wherein the conduit is placed in the gap of a transected nerve to guide the growth of the nerve through the conduit.

There remains an unmet need for treatments that can accelerate regeneration following peripheral nerve injury.

### SUMMARY OF THE INVENTION

The invention is based on the local delivery of a drug to a nerve. Delivering drugs locally to nerves using biomaterials provides a new approach to address the challenges in treating PNI. The inventors undertook extensive investigations of a number of biomaterials and identified several successful approaches to deliver a drug locally to a nerve, as described in the Examples. In various non-limiting aspects, the invention provides the following materials: (i) a drug-embedded membrane, such as an ethylene vinyl acetate (EVA) membrane, that can optionally be formed into tubes prior to administration to a patient; (ii) a drug-embedded membrane formed from polycaprolactone (PCL); and (iii) a drug-embedded electropsun nanofibrous polylactic-co-glycolic acid (PLGA) material. In some embodiments the drug-embedded material comprises or consists of a PolyLactic Acid-PolyCaprolactone copolymer (PLA/PCL).

According to one aspect of the invention, a nanofibrous material comprises a drug, wherein the nanofibrous material comprising the drug is for treating a peripheral nerve injury by delivering the drug locally to a damaged nerve. The nerve is typically mammalian, more typically human.

The material of the invention is typically flexible.

The material of the invention is typically able to be wrapped around a nerve. This typically requires that the material is flexible, so that it can be wrapped around a living nerve during surgery. The material can therefore typically be in the form of a sheet, membrane, bandage or wrap.

The material may be wrapped completely around a damaged section of a nerve. In this arrangement, the material covers all or substantially all of the outer circumference of at least a section of nerve, i.e. a particular length of nerve is covered. In one embodiment, two opposing edges of the material may meet or may overlap once applied to the nerve, forming a closed loop, tube or sheath that surrounds the nerve. In another embodiment, the material may be incompletely wrapped around the damaged section of the nerve. Incomplete or discontinuous wrapping may be useful where complete wrapping is not appropriate or possible. An incomplete wrap could, for example, form a sling around the damaged part of the nerve, or form a patch around part of the nerve.

In some embodiments, the material is a patch or dressing. The patch or dressing is suitable to be placed against (and in contact with) a nerve, or near a nerve but not in contact with it. The patch or dressing may, in some embodiments, be partially wrapped around the damaged part of a nerve. The patch or dressing is typically pliable and complements the nerve anatomy.

In other embodiments, the material is pre-formed into the required shape. This can be particularly useful to form a tube, sheath or cuff, that may find utility for example in being slid over a damaged (cut) end of completely transected nerve.

In certain embodiments, the material is biodegradable. Typically, a biodegradable biomaterial of the invention will dissolve or degrade in vivo once the drug has been delivered to the damaged nerve. In certain embodiments, the material is degraded within a year, within 6 months, within 150 days, or within 100 days of implantation.

As demonstrated in the Examples, a particular embodiment is provided wherein the material is a nanofibrous material that comprises or consists of polylactic-co-glycolic acid (PLGA). In further typical embodiments, the nanofibrous material such as PLGA is formed by electrospinning. The electrospinning may typically be coaxial electrospinning.

In some embodiments, the nanofibrous material comprises or consists of PLGA 50/50, wherein the lactide and glycolide monomers are present in a 50/50 molar ratio. PLGA 50/50 is shown in the Examples to provide a particularly useful release profile.

In some embodiments, the nanofibrous material comprises or consists of an L-Iactide/caprolactone copolymer, for example a 70/30 L-Iactide/caprolactone copolymer such as the commercially-available Purasorb® 7015, which has a CAS Registry number of 65408-67-5 and the chemical name (3S-cis)-3,6-dimethyl-1,4-dioxane-2,5-dione, polymer with 2-oxepanone. Such PLA/PCL materials are tested in FIG. 23, for example.

The nanofibrous material of the invention is typically thick enough to handle but thin enough to be able to wrap around a peripheral nerve. In some embodiments the material has a thickness between 10 and 1000 micrometres. In certain embodiments, the thickness is between approximately 50 and approximately 500 micrometres. This may be, for example, between 50 and 150 micrometres or between 75 and 125 micrometres.

In one embodiment, the nanofibrous material is 70/30 L-Iactide/caprolactone copolymer at a thickness of between approximately 50 micrometres and approximately 500 micrometres.

In one embodiment, the nanofibrous material is PLGA 50/50 at a thickness of between 50 micrometres and 500 micrometres.

In one embodiment, the nanofibrous material is 70/30 L-Iactide/caprolactone copolymer or PLGA 50/50 at a thickness of between 75 micrometres and 125 micrometres.

In addition to eluting the drug, the material of the invention may provide further advantages by acting as a physical support, in the manner of a bandage, for the damaged nerve that may aid recovery. In some embodiments, the material has a stiffness when in contact with the living nerve, that is similar to or greater than the stiffness of the nerve. In other embodiments, the material when in contact with the living nerve, has a stiffness that is less than the stiffness of the nerve.

The drug is typically embedded into or onto the material, and ideally allows for controlled and/or sustained release once implanted in vivo. In certain embodiments, the drug is incorporated into the material during the production of the material. In some embodiments the drug is completely encapsulated within the material. For example, when the material is electrospun PLGA, the drug can be incorporated into the PLGA in solvent prior to electrospinning.

In some embodiments the drug can be complexed with nanoparticles or microparticles, for example mesoporous silica nanoparticles (MSN). As shown in the examples, drug loading into nanoparticles such as MSN prior to combining the drug-loaded nanoparticles with the material of the invention, can improve the release profile of the biomaterial comprising the drug.

In some embodiments, the drug is a Non Steroidal Anti Inflammatory Drug (NSAID) or a PPAR agonist, optionally a PPARγ agonist. An exemplary drug is ibuprofen. Other drugs that may be used include drugs with neuro-regenerative function, for example dB-cAMP (dibutyryl cyclic adenosine monophosphate) or tacrolimus.

When the drug is ibuprofen and the material is electrospun PLGA, a suitable drug to polymer ratio (w/v of the drug in the PLGA/solvent solution) is from 1:2 to 1:25, for example 1:5 to 1:20, around 1:10, or around 1:7. When the drug is ibuprofen and the material is electrospun PLGA, the nanofibers may have an average diameter of between 0.5 and 1.5 μm, typically around 1 μm, for example around 0.92 μm as demonstrated in the Examples. The electrospun fibres are typically smooth, uniform and bead-free.

When the drug is ibuprofen and the material is 70/30 L-Iactide/caprolactone copolymer, a suitable drug to polymer ratio is 1:2 to 1:20, for example 1:10 as exemplified in FIG. 23.

In yet further embodiments, the material of the invention delivers a sustained-release of the drug to a living nerve, optionally wherein the drug is delivered at a sustained efficacious dose over a period of at least one week, at least two weeks, between 3 and 12 weeks, between 3 and 8 weeks, between 3 and 6 weeks, or between 6 to 8 weeks. When the drug is ibuprofen and the material is electrospun PLGA, the Examples demonstrate that drug release was controlled, exhibiting first order kinetics, over 1 week.

A second aspect of the invention provides a drug delivery system for delivering a drug locally to a nerve, wherein the system comprises a nanofibrous material. In certain embodiments of this aspect, the nanofibrous material comprises the drug and may be as defined above and elsewhere herein.

The nanofibrous material or drug delivery system of the first and second aspects may be packaged as a sterile single-use form. In further embodiments, a nanofibrous material or drug delivery system may be in a dry form, for example that can be stored for months or years until needed. In some embodiments, the invention provides a pre-made sterile drug-loaded material which can be stored stably in a dry form until administered during surgery.

A third aspect of the invention provides a method of treating a peripheral nerve injury in a patient in need thereof, comprising contacting a damaged nerve with a nanofibrous material comprising a drug, or a drug delivery system, according to the invention. Typically, the method of treating a peripheral nerve disease can comprise wrapping the material of the invention around the nerve.

A fourth aspect of the invention provides a device for placement around a damaged or injured peripheral nerve, or near a damaged or injured peripheral nerve, wherein the device comprises a drug-eluting nanofibrous material according to any of the previous aspects, that elutes the drug into the immediate vicinity of the damaged or injured peripheral nerve.

A fifth aspect of the invention provides a method of manufacturing a nanofibrous material comprising a drug, wherein the drug is incorporated into the nanofibrous material at an amount suitable to effect sustained release of an efficacious dose when in contact with a nerve in vivo. In one embodiment, the method comprises mixing the drug with the liquid material and its solvent, followed by electrospinning the mixture of drug and liquid material to form nanofibres. For example, PLGA can be dissolved into a suitable solvent (e.g. DCM), optionally at between 10 and 25% w/v, for example 17.5% w/v. The drug can be added to that solution and mixed, optionally to achieve a drug:polymer ratio of between 1:5 to 1:20, or 1:5 to 1:15, for example 1:7. The resulting solution can then be electrospun into fibres.

A sixth aspect of the invention provides a kit for preparing a nanofibrous material comprising a drug. The kit can comprise a coaxially electrospun nanofibrous PLGA sheet and ibuprofen. Alternatively, the kit may comprise a drug and one or more polymers, solvents and/or solutions for electrospinning into a nanofibrous PLGA sheet comprising the drug. The kit typically contains instructions for preparing the drug-containing material from the kit components, and may contain instructions for electrospinning the sheet. In certain embodiments, the nanofibre sheet has a size and dimensions suitable to wrap around a peripheral nerve and the ibuprofen is at a therapeutically-effective dose. In certain embodiments, the kit comprises the reagents needed to prepare the PLGA sheet and instructions for setting up the electrospinning parameters.

A seventh aspect of the invention provides a drug as described herein, typically a Non Steroidal Anti Inflammatory Drug (NSAID) or a PPAR agonist, optionally a PPARγ agonist, for use in a method of treating a peripheral nerve injury, wherein the NSAID or PPAR agonist is delivered locally using a material of the invention, optionally wherein material is a nanofibrous material as defined above and elsewhere herein.

An eighth aspect of the invention provides the use of a drug as described herein, typically a Non Steroidal Anti Inflammatory Drug (NSAID) or a PPAR agonist, and a nanofibrous material, in the manufacture of a medicament for the treatment of a peripheral nerve injury, optionally wherein the nanofibrous material is as defined above and elsewhere herein.

The materials, systems and methods of the invention are for use in treating a peripheral nerve injury. The PNI to be treated can include focal nerve injuries that require surgery. In various embodiments, the peripheral nerve injury can be a crush, a partial transection, a complete transection, a gap, or is caused by a neuropathy. The damaged nerve can also be a nerve that has been surgically repaired, optionally involving a graft such as an allograft, wherein the drug-loaded materials of the invention can be used to assist the recovery of the nerve from the surgery, or assist the engraftment and functional recovery.

### BRIEF DESCRIPTION OF THE DRAWINGS

FIG. 1: SEM images of blank and ibuprofen-loaded EVA membranes and tubes. The images were taken following cryogenic fracture of blank EVA membranes (a, b), ibuprofen-loaded EVA membranes (c, d), blank EVA tubes (e, f) and ibuprofen-loaded EVA tubes (g, h). Pores in the ibuprofen-loaded EVA membranes indicated with black arrows.

FIG. 2: XRD, TGA and DSC patterns of blank and ibuprofen-loaded EVA. XRD of the blank and ibuprofen-loaded EVA (a). TGA profiles (b). DSC profiles of the ibuprofen-loaded EVA (c), (d). Pure ibuprofen salt (black), blank EVA membranes (blue) and ibuprofen-loaded EVA membranes (red) (c, d).

FIG. 3: Drug release from ibuprofen-loaded EVA. Comparison of increasing concentration drug loads (a) of 0.5%, 1%, 2% and 4% (w/v) ibuprofen loading into flat sheet membranes (b) and manufactured tubes. Comparison of release from an EVA membrane and tube (c).

Representation of an EVA tube (d) and comparison of release from the internal and external surface of the EVA tube (e). N=3, mean±SEM.

FIG. 4: SEM images of blank, ibuprofen-loaded PCL membranes and ibuprofen-loaded MSN embedded in PCL. Blank PCL membranes (a) ibuprofen loaded PCL membranes (b) and ibuprofen-loaded MSN embedded in PCL (c). Drug release from ibuprofen-loaded PCL (d) and ibuprofen-loaded MSN embedded in PCL (e). The initial drug load is shown by the dotted line. N=3, mean±SEM.

FIG. 5: SEM images of blank and ibuprofen or sulindac sulfide loaded electrospun PLGA nanofibres. Blank PLGA nanofibres (a, b) and ibuprofen-loaded PLGA nanofibres (c, d). The nanofibres presented different surface appearances under different storage conditions; (e, g) at 4° C. and (f, h) at 27° C. for 7 days.

FIG. 6: XRD, TGA and DSC patterns of blank and ibuprofen-loaded PLGA nanofibres. XRD of the blank and ibuprofen-loaded PLGA (a). TGA profiles (b). DSC profiles of the ibuprofen-loaded PLGA nanofibres (c, d). Pure ibuprofen sodium salt (black), blank PLGA membranes (blue) and ibuprofen-loaded nanofibres membranes (red) (c, d).

FIG. 7: Drug release from ibuprofen and sulindac sulfide-loaded electrospun PLGA nanofibres. Ibuprofen loaded nanofibres (a) and sulindac sulfide nanofibres (b). N=3, mean±SEM.

FIG. 8: Axon number in a transection injury model with an implanted ibuprofen-loaded EVA tube. Surgically implanted EVA tubes (a) and harvesting tubes at 21 days post injury (b). Scale bar=5 mm. Axons were quantified by counting the number of neurofilament-positive cells in the proximal stump and distal stump at 21 days post injury. The number of axons in the distal stump increased in the group with an implanted ibuprofen-loaded EVA tube in comparison to the blank EVA tube (c, d). Also the number of axons in the distal nerve stump exceeded those in the proximal stump (c). Micrographs are 10 μm transverse sections showing neurofilament positive neurites (e). Scale bar=100 μm. N=6, mean±SEM for each condition. Two-way ANOVA (a), and two-tailed t-Test (b), *p<0.05.

FIG. 9: Quantitative analysis of the number (a) and diameter (b) of blood vessels by RECA-1 (green). Immunostained 10 μm transverse sections from the proximal and distal stumps (c). Scale bar=100 μm. N=6, mean±SEM for each condition, One-way ANOVA with Tukey's post hoc test, *p<0.05. Box plots show the distribution of blood vessel diameter with boxes extending from the max to min, + indicates mean.

FIG. 10: Axon number in a crush injury model with implanted ibuprofen or sulindac sulfide-loaded PLGA nanofibres. Surgically implanted and harvesting at 28 days post injury of ibuprofen-loaded PLGA wraps (a) and sulindac sulfide-loaded PLGA wraps (b). Scale bar=5 mm. Axons were quantified by counting the number of neurofilament-positive cells in the proximal and distal stumps. The number of axons in the distal stump increased in the groups with implanted ibuprofen (c, d) and sulindac sulfide-loaded (e, f) PLGA nanofibers in comparison to their corresponding control groups at 28 days. N=3 (sulindac sulfide), N=4 (ibuprofen), mean±SEM for each condition. Two-way ANOVA (c, d), and two-tailed T-Test (e, f), no significance.

FIG. 11: Von Frey following a crush injury treated with ibuprofen and sulindac sulfide-loaded PLGA nanofibres. The threshold response returned to baseline quicker in the ibuprofen (a) and sulindac sulfide (b) treatment groups in comparison to the control groups after 28 days. N=3 (sulindac sulfide), N=4 (ibuprofen), mean±SEM, Multiple T-tests between the injured groups at each time point *p<0.05.

FIG. 12: SSI following a crush injury treated with ibuprofen and sulindac sulfide-loaded PLGA nanofibres. A significant difference in the SSI was seen between the ibuprofen treatment groups and the control (a) but not in the sulindac sulfide (b) treatment group. N=3 (sulindac sulfide) N=4 (ibuprofen), means±SEM, multiple T-tests, *p<0.05.

FIG. 13: Electrophysiological evaluation of a crush injury treated with ibuprofen or sulindac sulfide-loaded PLGA nanofibres at 28 days post injury. The CMAP was significantly higher in the treatment group at 28 days following ibuprofen treatment (a) but not with sulindac sulfide treatment (b). No difference was seen in the latency with either drug treatment (c, d). The stimulus intensity was lower in the ibuprofen treatment group in comparison to the control but was not significantly significant (e) and there was no difference seen with sulindac sulfide treatment (f). N=3 (sulindac sulfide), N=4 (ibuprofen), means±SEM, Two-way ANOVA, *p<0.05.

FIG. 14: Vasculature changes following a crush injury treated with ibuprofen and sulindac sulfide-loaded PLGA nanofibres. Quantitative analysis of the number and diameter of blood vessels in ibuprofen-loaded PLGA (a, c) and sulindac sulfide-loaded PLGA (b, d) at 28 days post injury using RECA-1 (green). Immunostained 10 μm transverse sections from the proximal and distal stumps (e). Scale bar=100 μm. N=3 (sulindac sulfide), N=4 (ibuprofen), mean±SEM. One-way ANOVA with Tukey's post-hoc test, *p<0.05. Box plots show the distribution of blood vessel diameter with boxes extending from the max to min, + indicates mean.

FIG. 15: Functional evaluation of a transection injury treated with ibuprofen-loaded EVA following 21 days treatment. No differences were seen between the control and ibuprofen treated groups in the functional outcome measure; gastrocnemius muscle mass (a), von Frey (b), static sciatic index (c) and electrophysiology (d-f).

FIG. 16: Schematic overview of certain aspects of the technology.

FIG. 17: The effect of polymer type on drug release profile. All formulations were loaded 1:10 w/w with ibuprofen.

FIG. 18: The effect of PLGA composition and drug loading on release profile.

FIG. 19: The effect of PLGA composition and drug loading on release profile—first 6 hours.

FIG. 20: PLGA 50/50 (Mw 44,000) scaffolds after (a) 3-weeks, and (b) 5-weeks incubation. On the left [L] blank fibres, on the middle [M] ibuprofen-loaded 1:20 (w/w) fibres and on the right [R] ibuprofen-loaded 1:10 (w/w) fibres. Loaded samples [M and R] are shown to have degraded further when compared with the blank [L] scaffolds. A clear progression can be seen from 3 to 5 weeks incubation.

FIG. 21: Material handling of PLGA 50/50 and PLGA 75/25.

FIG. 22: The effect of fibre thickness on handling properties tested in the in vitro sciatic nerve model. Fibres of varying thickness were produced by electrospinning different volumes of polymer solution— 1.5 mL (panels a and c) or 2.25 mL (panels b and d). Thicker fibres showed poorer handling properties.

FIG. 23: The effect of fibre thickness on handling properties tested in the in vivo rat model. Fibres of varying thickness were produced by electrospinning different volumes of polymer solution— 1.5 mL (panels A and C) or 2.25 mL (panels B and D). Handling properties of both materials allowed successful implantation around the rat sciatic nerve (A, B). No fibrosis was observed after 21 days in vivo, and materials could easily be removed from the nerve (C,D).

FIG. 24: Additional images showing the effect of fibre thickness on handling properties tested in the in vitro sciatic nerve model. Fibres of varying thickness were produced by electrospinning different volumes of polymer solution. Thicker fibres showed poorer handling properties.

FIG. 25: a) SEM images of PDLG 5010 core-shell electrospun fibres loaded with dB-cAMP, b) histogram showing the diameter distribution of fibres which was calculated using ImageJ (n=100 measurements from 3 different samples) and c) cumulative drug release from fibres. Data expressed as mean+1- SE (n=3, 1 experiment).

FIG. 26: A: Zero-order type in vitro tacrolimus release from coaxially electrospun PCL fibre sheet (data are n=2 mean±SD). B: SEM images of fibre sheet used in release study (left, scale bar 10 μm), and recent formulations optimised for morphology (centre, scale bar 50 μm, and right, scale bar 10 μm). C: PCL fibre sheets successfully implanted at site of a rat sciatic nerve transplant. D: Micrographs of immunofluorescence showing increased T cell infiltration in nerve allografts which is reduced by local delivery of tacrolimus. E: Quantification of CD4 (left) and CD8 (right) T cells per mm² within longitudinal graft sections (n=4 per group, all data are means ±SEM). F: Weight gain in study animals indicating no side effects of tacrolimus.

### DETAILED DESCRIPTION OF THE INVENTION

The present inventors have created a delivery platform using biomaterials to deliver a drug locally in a controlled manner for treating peripheral nerve injury (PNI). Advantages of this invention include that nerve regeneration will proceed at a faster rate using this treatment, reducing the delay that leads to tissue atrophy and improving recovery of function, reducing the extent and duration of disability for patients, providing a minimally-invasive medicinal treatment where currently none is available, and avoiding side effects associated with systemic long-term drug administration. This method of drug delivery also reduces poor patient compliance by removing the need for patients to remember to take medication multiple times a day for a long period of time.

In certain embodiments, the invention relates to drug-eluting biomaterials for promoting nerve regeneration by eluting a drug that is incorporated into or onto the biomaterial, into the immediate vicinity of a damaged nerve.

In some embodiments, the biomaterials of the invention may be characterised as a nerve wrap or a nerve bandage.

The invention is directed to materials for the treatment of a peripheral nerve injury (PNI). The physiology, in particular the size and local tissue environment, of peripheral nerves means that materials suitable for treating spinal cord injuries (SCI) will typically not be suitable for treating PNI, in particular for wrapping around a peripheral nerve. Furthermore, a conventional nerve repair conduit that provides a physical track or path through which the cut end of a transected nerve grows, is very different from a nerve wrap of the invention that delivers drug locally to the external surface of the nerve. In particular, conventional nerve conduits cannot be used for crush injuries.

Agonists of the peroxisome proliferator-activated receptor gamma (PPARγ) show beneficial effects on PNI in animal studies. However, systemic administration of PPARγ agonists has side effects that preclude sustained systemic dosage in patients recovering from nerve injury. The invention is based at least in part on the realisation that local administration of PPARγ agonists to the site of nerve injury, typically using electrospun biomaterials, can accelerate nerve regeneration. This opens up the possibility for a new therapeutic product, a drug-eluting nerve wrap, for implantation during nerve surgery to accelerate regeneration and improve functional outcomes for patients.

The invention also provides a controlled-release biomaterial formulation that will deliver drugs, such as PPARγ agonists, locally at a sustained relevant dose. The electrospinning approach can be used to produce drug-eluting material for detailed characterisation and clinical validation.

The invention therefore provides an implantable material that can be placed at the site of a nerve injury in order to release a drug in a controlled manner over a sustained period of time in order to accelerate nerve regeneration, reducing the delay in muscle reinnervation and thus preventing muscle wastage and improving restoration of function.

Biomaterials suitable for implantation at the site of nerve injury include synthetic polymers and natural materials that would biodegrade over time, with the potential to provide controlled-release of drug¹⁰. Controlled-release systems using degradable and non-degradable biomaterials to deliver drugs have demonstrated effectiveness in other indications¹⁰⁻¹² and can be tested for application in a neural environment. Most drug-release materials are relatively short-acting, however for nerve injury there is likely to be benefit in releasing pro-regenerative drugs over a sustained period of weeks to maximise benefit throughout the regeneration period.

In certain embodiments described herein, the local delivery of a drug (for example ibuprofen or sulindac sulfide) to a damaged or injured nerve is provided using a material of the invention. In particular exemplified embodiments, the material can be selected from a polycaprolactone (PCL) material or a polylactic-co-glycolic acid (PLGA) material. Polylactic acid/polycaprolactone (PLA/PCL) copolymers are also provided in some embodiments.

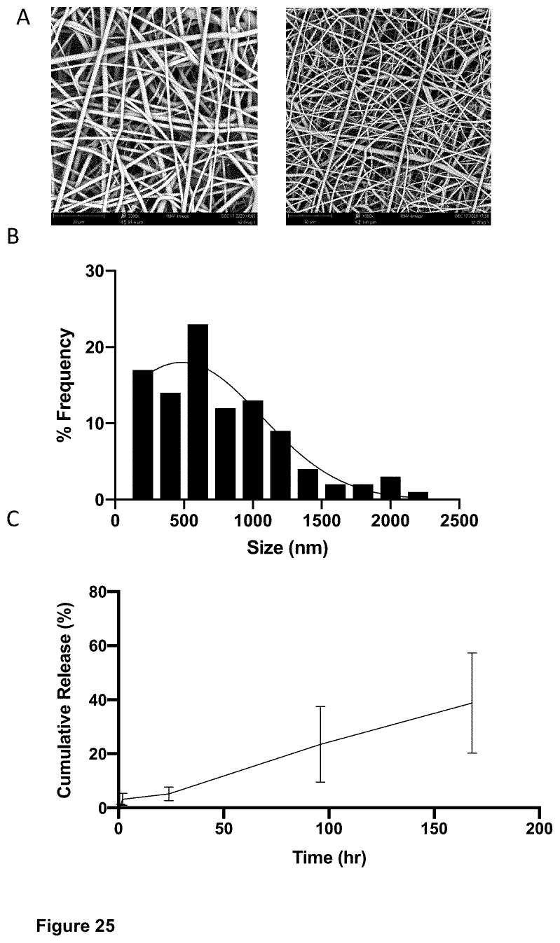

In certain embodiments, the drug stimulates neural growth and/or proliferation, for example dB-cAMP (dibutyryl cyclic adenosine monophosphate). This is shown to be sustainably released using a biomaterial of the invention, in FIG. 25.

Another drug with pro-regenerative effects that can be eluted from a biomaterial according to the invention, is tacrolimus. The Examples also report data showing that tacrolimus, a drug with pro-regenerative effects on nerves, can also be delivered locally using the biomaterials of the invention. Tacrolimus is also immunosuppressive, and in one embodiment can be used to improve allograft acceptance and simultaneously accelerate regeneration. The biomaterial of the invention comprising tacrolimus can therefore be used to wrap around a nerve allograft to enhance engraftment and recovery. The data show that a beneficial structure and release profile is obtained using tacrolimus and a biomaterial of the invention, and also that it causes local immunosuppression in vivo.

In certain embodiments, a nanoparticle or microparticle can be used to encapsulate the drug which is then trapped within the biomaterial fibres or sheets. The use of trapped nano/microparticles is therefore provided. An example of a nanoparticle is a mesoporous silica nanoparticle (MSN) and others will be apparent to the skilled person. Nanoparticles can be made according to methods known in the art. In one embodiment, the nanoparticles can be produced by electrospraying, for example electrospraying PLGA with appropriate voltage and collection parameters. Core-shell electrosprayed nanoparticles made from PCL and/or PLGA and that encapsulate the drug are therefore provided in some embodiments.

In some embodiments, the drug-loaded polymers can be prepared as sheets using solvent casting, or as nanofibers using electrospinning. This latter nanofibre electrospinning approach involves the application of a voltage to an extruded polymer solution so as to result in the generation of fibres, which can then be used for numerous delivery and biomaterial applications. The use of these fibres as a drug-loaded sheath is provided as part of the invention, to provide both physical support and prolonged release of the therapeutic agents.

EVA is a non-degradable polymer approved for use in a range of clinical applications to deliver drugs such as hormonal contraception, pilocarpine for glaucoma and buprenorphine for opioid addiction¹¹⁻¹³. PCL is a biocompatible aliphatic polyester used clinically for hormonal contraceptive implants and it has been extensively investigated as a nerve conduit for PNI repair ^{14, 15}.

Currently, a Phase I clinical trial is recruiting participants to evaluate the use of PCL nerve conduits as a therapy in sensory digital nerve surgery¹⁶. An attempt at ibuprofen loading into PCL has been reported in relation to development of nerve conduits although the effects on neuronal regeneration were not tested in vitro or in vivo¹⁷. PLGA has been used to deliver growth factors, hormones and drugs in experimental PNI treatment¹⁸. MSNs are versatile drug-delivery materials ¹⁹ and previous studies using ibuprofen have shown a high loading content of the poorly soluble drug and a release rate of 96.3% ²⁰, making them another promising material for investigation in PNI.

The Examples below demonstrate that NSAIDs can be delivered locally to improve regeneration following PNI, thus overcoming the limitations associated with systemic administration. A range of drug/material combinations are tested in vitro to establish stability and drug-release parameters. Each tested material and formulation is provided as an aspect of the invention. Controlled-release formulations of NSAIDs in polymeric sheaths of EVA and PLGA were then positioned around a nerve transection and crush injury respectively in a rat PNI model, increasing neurite growth and supporting the hypothesis that locally delivered NSAIDs might be of benefit.

Electrospun nanofibrous materials can be formulated to release the drug at the repair site, maintaining an efficacious local dose around the nerve but avoiding the side-effects associated with systemic administration of certain drugs, for example as seen for PPARγ agonists. Nerves are soft tissue structures which can be affected by local mechanical disruption (e.g. through being surrounded by stiff materials), so the material can be a thin nanofibrous membrane resembling ‘tissue paper’ which when dry can be stored and handled then when placed adjacent to the nerve will integrate with no mechanical mismatch or local disruption. Once the drug release phase is complete, the material will gradually degrade into harmless products that will be cleared naturally as the injury site heals.

The Examples demonstrate an electrospun polymer-based ibuprofen-delivery material that showed positive effects in vivo. In certain embodiments, the invention provides an implantable electrospun PLGA wrap that delivers a regeneration-enhancing drug to a repaired nerve at an optimal dose over a number of weeks, for example 3-6 weeks.

Local administration of the PPARγ agonist ibuprofen can be highly effective in accelerating nerve regeneration and functional recovery in animal models without any adverse side effects. Ibuprofen delivered using osmotic minipumps, non-degradable polymer materials and degradable electrospun polymers has been tested in vitro and in vivo (Rayner, M. L. D., et al., Developing an In Vitro Model to Screen Drugs for Nerve Regeneration. Anat Rec (Hoboken), 2018. 301(10): p. 1628-1637). Additionally, in vitro modelling showed a new link between PPARγ affinity and regeneration support, leading to an alternative PPARγ agonist sulindac sulphide being tested successfully using an electrospun polymer material for delivery.

The Examples (FIG. 25) also show the successful loading and sustained release of dibutyryl cyclic adenosine monophosphate, using an electrospun PLGA nanofibrous material. This drug is not a PPAR gamma agonist but has been shown to have a positive effect on nerve regeneration.

The Examples also report data showing that tacrolimus, a drug with pro-regenerative effects on nerves, can also be delivered locally using the biomaterials of the invention.

Previous systemic dosing studies using ibuprofen in animals showed that sustained treatment over 3-6 weeks results in improved nerve regeneration. Coaxial electrospinning of poly-ε-caprolactone (PCL) has been used to produce nanofibrous materials that exhibit zero-order sustained release over timescales suitable for implantation (Angkawinitwong, U., et al., Electrospun formulations of bevacizumab for sustained release in the eye. Acta Biomater, 2017. 64: p. 126-136), providing a system which can be tuned to provide an optimal controlled-release formulation for use in this technology.

In some embodiments, the invention uses biodegradable synthetic polymers known for use in medical applications and suitable for GMP manufacture and regulatory approval. Used separately or as blends or composites, they can be tuned to achieve specific mechanical properties, degradation rates and drug loading/release profiles. This versatility and broad acceptance combined with the data in the Examples below supports this choice of material to form drug-eluting materials, for example nanofibrous materials such as electrospun PLGA, for nerve applications.

The material of the invention releases a drug (e.g. PPARγ agonist) locally in a sustained and controlled manner. In particular, it has been shown that coaxial electrospinning provides zero-order release kinetics. A selection of electrospun nanofibrous materials can be generated based on materials including poly(lactic-co-glycolic acid) (PLGA) or PCL, incorporating specific PPARγ agonists optionally selected from ibuprofen, sulindac sulphide and diclofenac. Material formulations can be refined and selected to provide appropriate release kinetics, stability and handling properties, as will be apparent to the skilled person.

Electrospun nanofibrous materials can be generated to incorporate a drug of interest. The drug may optionally be one or more of ibuprofen, sulindac sulphide, or diclofenac. Other suitable drugs include dibutyryl cyclic adenosine monophosphate and tacrolimus.

A monolithic PLGA formulation can also be made. Coaxial electrospun formulations using PCL, PLGA, or a combination, may also be used. A core/shell PLGA is another formulation. Monolithic PCL is yet another alternative material. Formulations can be refined and selected to provide appropriate release, stability and handling properties. Release kinetics can be assessed over time, for example over 21 days (e.g. using UV-vis spectroscopy to quantify drug release into aqueous solution), nanofibre stability can be assessed using accelerated aging studies (e.g. dry mass of material, presence of monomers/oligomers/drug in solution), and mechanical properties can be tested using tensile and compressive dynamic mechanical analysis.

In some embodiments, the invention provides drug-loaded nanofibrous Poly Lactic-co-Glycolic Acid (PLGA) as a local drug delivery platform to treat peripheral nerve injury.

The use of nanofibrous PLGA sheets as a local delivery system allows local administration of drugs at the site of nerve injury. Particular advantages of using this biomaterial preparation include (1) PLGA is biodegradable and widely accepted for use in other clinical applications, (2) the nanofibrous sheet formulation (generated by electrospinning) provides ideal physical handling properties for wrapping around an injured nerve, (3) it can be loaded with a range of small molecules that can improve nerve regeneration when delivered locally, (4) dose, rate and duration of drug release can be precisely controlled through controlling the loading and formulation of the material, and also the lactide-glycolide ratio in PLGA can be altered to adjust the degradation time (5) local delivery using a degradable material provides for the first time the opportunity to use efficacious drugs that would otherwise be avoided due to side effects associated with systemic administration.

Patients with PNI typically have their nerves exposed during surgical repair, so wrapping a drug-loaded material around the repaired nerve at that stage is no more invasive than the repair surgery itself. Furthermore, the use of a degradable material means that there is no need for a second procedure to remove the device. Using this approach to deliver ibuprofen improves the rate and extent of regeneration in a rat nerve injury model using multiple outcome measures. Other therapeutic agents can be used in this system, including other small molecules and drugs similar to ibuprofen.

### Examples

Summary—Controlled Local Release from Biomaterials to Treat Peripheral Nerve Injury

Local controlled-release drug delivery systems could potentially address the challenges of peripheral nerve injury, particularly through the use of biomaterials that can be implanted at the repair site during the microsurgical repair procedure. In order to test this concept, this study used various biomaterials to deliver ibuprofen or sulindac sulfide locally in a controlled manner in a rat sciatic nerve injury model.

Following characterisation of release parameters in vitro, ethylene vinyl acetate (EVA) tubes or polylactic-co-glycolic acid (PLGA) wraps, loaded with of PPARγ agonists (ibuprofen or sulindac sulphide), were placed around directly-repaired nerve transection or nerve crush injuries in rats. Ibuprofen caused an increase in neurites in distal nerve segments and improvements in functional recovery in comparison to controls with no drug treatment. This study showed for the first time that local delivery of ibuprofen using biomaterials improves neurite growth and functional recovery following PNI.

Keywords:

Nerve regeneration; Non-steroidal anti-inflammatory drugs (NSAIDs); Local drug delivery; Biomaterials; Peroxisome proliferator-activated receptor gamma (PPARγ); Peripheral nerve injury (PNI).

Material and Methods

All materials were supplied by Sigma-Aldrich unless otherwise stated.

Drug Loading into Biomaterials

Drug embedded EVA membranes were manufactured by dissolving 2 g EVA co-polymer beads and 1%, 2 % and 4% (weight per volume of solvent) ibuprofen in 20 mL chloroform. Once the drug and the polymer had fully dispersed, the solution was added to a rectangular 83 mm×63 mm glass mould and dried at room temperature for 24 h. The membrane was then cut into 5 mm×12 mm×0.5 mm flat sheets for characterisation and implantation studies. EVA sheets with no embedded drug were manufactured using the same procedure and used as control samples.

EVA membranes were manufactured into tubular-shaped constructs for implantation by wrapping the EVA sheet around a 19G needle and fusing the edge together with chloroform. The tubes were dried at room temperature and removed from the needle furnishing a tube with dimensions 5 mm×12 mm and 1.5 mm inner diameter.

Drug embedded PCL membranes were manufactured by dissolving 100 mg ibuprofen in 5 mL chloroform then adding 5 g PCL beads to the solution and stirring for 18h at room temperature. The homogenous polymer solution was poured into a circular Teflon mould (Ø77 mm) and dried for 2-3 h at room temperature to allow the solvent to evaporate. Once the solvent was removed the membrane was cut into smaller sheets (7 mm×12 mm×0.4 mm) for characterisation. PCL membranes without drug were prepared using the same procedure and used as control samples. 100 mg of mesoporous silica nanoparticles (MSN) (kindly donated by Ahmed El-Fiqi, UCL Eastman Dental Institute)¹⁹ were dispersed in 10 mL ibuprofen solution (2% w/v ibuprofen sodium salt prepared in distilled water) and incubated for 6 hours at 37° C. to allow drug loading. The resulting solution was centrifuged at 400 g for 5 minutes to obtain a pellet of MSN. They were then left to dry at 37° C. overnight. During this time the MSN aggregated and formed clumps so the pellet was ground to re-obtain a powder. Drug loading into the MSN was determined by measuring the quantity of ibuprofen remaining in the stock solution using UV-Vis spectrophotometry (Unicorn UV500).

A PCL membrane with ibuprofen-loaded MSN was made by dispersing the MSN in 5 mL chloroform (1% or 2% of ibuprofen-loaded MSN, which corresponded to 50 mg and 100 mg of MSN, respectively). 500 mg of PCL beads were added to this solution and the mixture was stirred at room temperature for 18 h. The MSN loaded PCL was manufactured using the same method.

Electrospinning of Poly (lactic-co-glycolic acid) (PLGA)

Poly (lactic-co-glycolic acid) (PLGA) (Corbion Purac with molecular weight (MW) of 96,000 Grade: PURASORB PDLG 7507 ratio 75:25) nanofibers were fabricated by electrospinning using a Spraybase® electrospinning instrument)(Spraybase®. The PLGA 17.5% w/v was dissolved in dichloromethane (DCM) and stirred gently for 45 min. Then 2.5% w/v ibuprofen or 1% w/v sulindac sulfide was added and stirred for another 45 min to achieve a drug to polymer ratio of 1:7, or 1:17 respectively. The ratios were selected based on complete encapsulation of the drug within the polymer. The solution was loaded into a 10 mL syringe with a diameter of 14.43 mm to be ejected through a 0.7 mm diameter needle. The flow rate and voltage used to stabilize the jet were 1 mL/h and 10-11 kV, respectively. The fibres were collected on aluminium foil at a distance of 12.5 cm. Similar parameters were used to prepare blank fibres with no drug embedded. The drug encapsulation efficiency and drug loading was determined by dissolving the fibres in 20 mL of acetonitrile for 4 h. The resulting solutions were analysed using UV-Vis spectroscopy.

Material Characterization

Scanning electron Microscopy (SEM)

The morphology and particle size of the polymeric samples were characterized using SEM (Philips XL30 FEG or FEI Quanta 200F) at 5 kV. Samples were mounted onto metal specimen stubs, using double-sided adhesive tape, vacuum coated with a platinum film and then viewed and imaged. The particle size and porous structure of MSN were characterised using TEM (Philips CM12) operated at 80 kV.

Drug release

Drug release was determined by incubating the material in 1 mL distilled water at 37° C. The 1 mL solution was collected at fixed time points (1 h, 2 h, 3 h, 4 h and then every 24 h) and replaced with 1 mL of fresh distilled water. The solution collected was analysed with a UV-Vis spectrophotometer (Unicam UV500) at a wavelength of 263 nm.

X-ray Diffraction (XRD)

XRD spectra were acquired using Rigaki Miniflex 600 X-Ray Diffractometer with measurements taken within an angle range of 3-60° at 0.02° increments. The instrument was supplied with Cu Kα radiation at 40 kV and 15 mA.

Differential Scanning Calorimetry (DSC)

DSC was conducted by loading a pan with 55 mg of material into a differential scanning calorimeter (DSC 2000 TA) and the glass transition temperature was determined by increasing the temperature at a rate of 10° C./min within the range of 0-170° C.

Thermogravimetric Analysis (TGA) 5 mg of sample was loaded into TGA running pans which were subsequently loaded onto a Thermogravimetric analysis machine (Discovery series, Universal V4.5A TA Instruments) and the sample was heated at a rate of 20° C./min to a maximum of 400° C. The nitrogen purge was set at 25 mL/min and balance purge flow of 10 mL/min.

Surgical Nerve Injury Models

All surgical procedures were performed in accordance with the UK Animals (Scientific Procedures) Act (1986), the European Communities Council Directives (86/609/EEC) and approved by the UCL Animal Welfare and Ethics Review Board. Adult male Wister rats (250-300 g) (Charles River) were deeply anaesthetised by inhalation of isoflurane, and the left sciatic nerve was exposed at mid-thigh level then subjected to either a transection or a crush injury. A transection injury with a primary repair was conducted by making a cut through the entire nerve (1.5 cm distal of the top of the femur) and the proximal and distal stumps were re-connected using two 10/0 epineurial sutures, one on each side of the nerve at each stump. EVA tubes pre-loaded with vehicle control or drug treatment were placed around the injury site like a cuff by threading a nerve stump through the tube between transection and repair, then sliding the tube over the repair after suturing. The crush injury was achieved by applying a consistent pressure with a pair of sterile TAAB tweezers type 4 closed fully on the same point of the nerve (1.5 cm distal of the femur) for 15 s. This was repeated twice more in the same location with the tweezers positioned perpendicular to the nerve and rotated through 45° between each crush application²¹. A 10/0 epineurial suture (Ethicon) was used to mark the location of the crush. The ibuprofen- or sulindac sulfide-loaded PLGA was wrapped around the injury site as a cuff.

The overlying muscle layers were closed using two 4/0 sutures (Ethicon) and the skin was closed using stainless steel wound clips. Animals were allowed to recover and were maintained for 21 or 28 days then culled using CO₂ asphyxiation and the repaired nerves were excised under an operating microscope and immersion-fixed in 4% (w/v) paraformaldehyde in PBS at 4° C. The gastrocnemius muscles on both the repaired and contralateral side were separated from the soleus muscle and stored in 4% PFA on ice and weighed immediately.

Electrophysiology

After 21-28 days animals were anaesthetised using isoflurane and nerve function was assessed using electrophysiology (Sapphire 4ME system) by comparing the repaired nerve to the contralateral undamaged nerve in each animal. Electrodes (Natus) were attached to the animal; a grounding electrode was placed onto the tail of the animal and a reference electrode was placed above the hip bone. A stimulating electrode (Neurosign Bipolar Probe 2×100 mm×0.75 mm electrode) was placed against the proximal nerve 2 mm above the injury site and a monopolar recording needle (Ambu® Neuroline concentric) was placed into the gastrocnemius muscle. The distance between the stimulating and recording electrodes was standardised. The nerve was stimulated using a bipolar stimulation constant voltage configuration and the muscle response recorded. The stimulation threshold was determined by increasing the stimulus amplitude in 0.1 V steps (200 ρs pulse), until both a supramaximal, stimulus-correlated compound muscle action potential (CMAP) was recorded and a significant twitch of the animal's hind paw could be seen. The CMAP amplitude (mV) was measured from baseline to the greatest peak and the latency was measured from the time of stimulus to the first deviation from the baseline. CMAPs were recorded in triplicate for both the injured nerve and contralateral control nerve in each animal.

Von Frey Sensory Assessment

The animals were placed on a grid and von Frey filaments (0.008 g−300 g) were applied through the underside of the grid to stimulate the centre of the animal's hind paws. A response was determined by the retraction of the animal's paw following the filament stimulus. The threshold response was recorded by decreasing the stimulus until no response was detected.

Static Sciatic Index (SSI)

The animal's hind paws were imaged and the toe spread factor (TSF), between the 1^{st} and 5^{th} toe, and the intermediary toe spread factor (ITSF), between the 2^{nd} and 4^{th} toe, were measured and equation 1 was used to calculate SSI²².

SSI=(108.44×TSF)+(31.85×ITSF)−5.49 Equation 1

TSF=(TS_{injury}−TS_{control})/TS_{control} ITSF=(ITS_{injury}−ITS_{control})/ITS_{control}

Cryo-Sectioning

Following fixation the nerve samples were incubated in 30% sucrose overnight and underwent subsequent snap freezing in 1:1 FSC 22 Frozen Section Media (Leica) and 30% sucrose. Transverse sections (10 μm) were prepared from the proximal and distal stumps 5 mm from the injury site, using a cryostat (Leica CM1860). The sections were adhered to glass slides (Superfrost™ Plus, Thermo Fisher Scientific) for histological analysis.

Immunohistochemistry

Nerve sections were washed in immunostaining buffer (PBS together with 0.2% Triton-X, 0.002% sodium azide and 0.25% bovine serum albumin before the addition of serum (Dako) to block non-specific binding (1: 20 dilution). After 30 mins the blocking serum was removed and sections were incubated with neurofilament-H (Eurogentec, 1:1000) or RECA-1 (Millipore, 1:100) primary antibody diluted in immunostaining buffer overnight at 4° C. The sections were washed with immunostaining buffer before addition of the Dylight 549 or 488 secondary antibody (Vector Laboratories, 1:400) and incubation at room temperature for 45 mins. Sections underwent a final wash with immunostaining buffer before mounting with Vectashield Hardset mounting medium with DAPI (Vector Laboratories).

Image Analysis and Quantification

Tile scans were used to capture high-magnification (×20) micrographs from the entire nerve cross-section using a Zeiss LSM 710 confocal microscope and images were analysed using Volocity™ 6.4 (PerkinElmer) running automated image analysis protocols to determine the number of neurofilament-immunoreactive neurites in each transverse nerve section. Blood vessel and macrophage analysis was conducted from entire nerve sections using fluorescence microscopy (Zeiss Axiolab A1, Axiocam Cm1) and blood vessel diameter was measured using ImageJ software²³.

Statistical Analysis

Normality tests were conducted on all data to determine appropriate statistical tests, and one-way analysis of variance (ANOVA) or t-tests were performed, as data followed a normal distribution. A one-way ANOVA was followed by a Tukey or Dunnett post hoc test. For all tests, *p<0.05, **p<0.01, ***p<0.001 and ****p<0.0001 were considered to be significant.

Results

Characterisation of Materials

Ethylene Vinyl Acetate (EVA)

Scanning electron micrographs indicated that pore size changed as a result of drug loading with pores ˜5 μm in the control and ˜25 μm in the ibuprofen-loaded material (FIG. 1). Crystals of drug were visible within the pores (FIG. 1(c), (d)). The composition of EVA tubes with and without ibuprofen were also analysed using SEM following cryogenic fracture in liquid nitrogen (FIG. 1(e-h)). The tubes of the required diameter were formed by overlapping and fusing the edges of the membrane (FIG. 1(e), (g)).

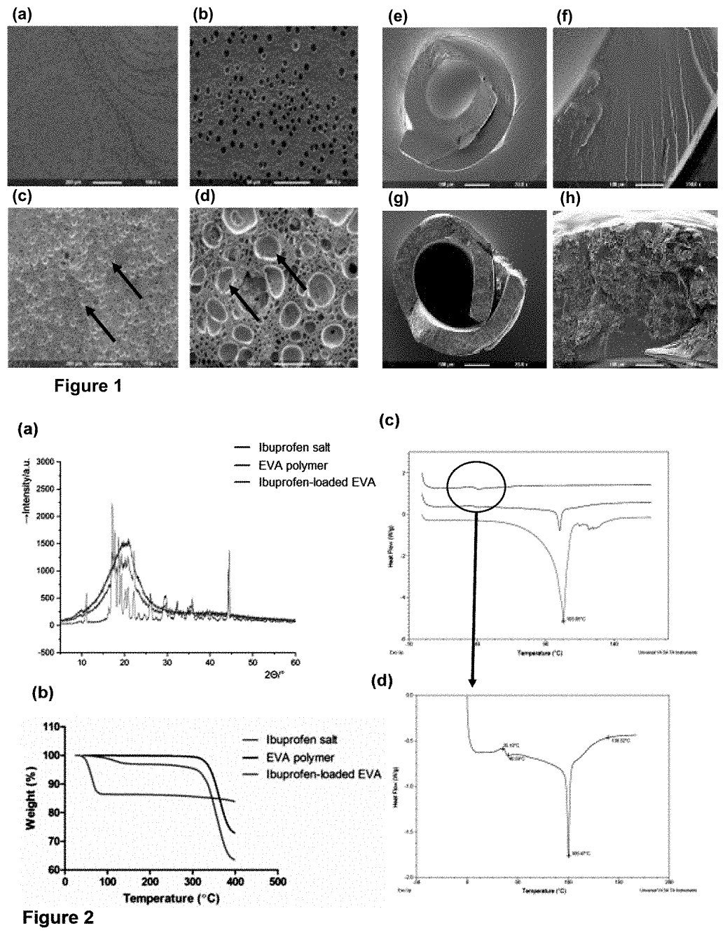

X-ray diffraction (XRD) analysed the physical properties of the material. The drug loaded material displayed a similar profile to pure ibuprofen salt and EVA suggesting that the drug and polymer hadn't mixed completely (FIG. 2(a)). From 15° to 25°, the ibuprofen loaded EVA membrane presented small sharp reflections at the same position as those seen with pure ibuprofen, indicating that some ibuprofen presented as crystals.

Drug Release

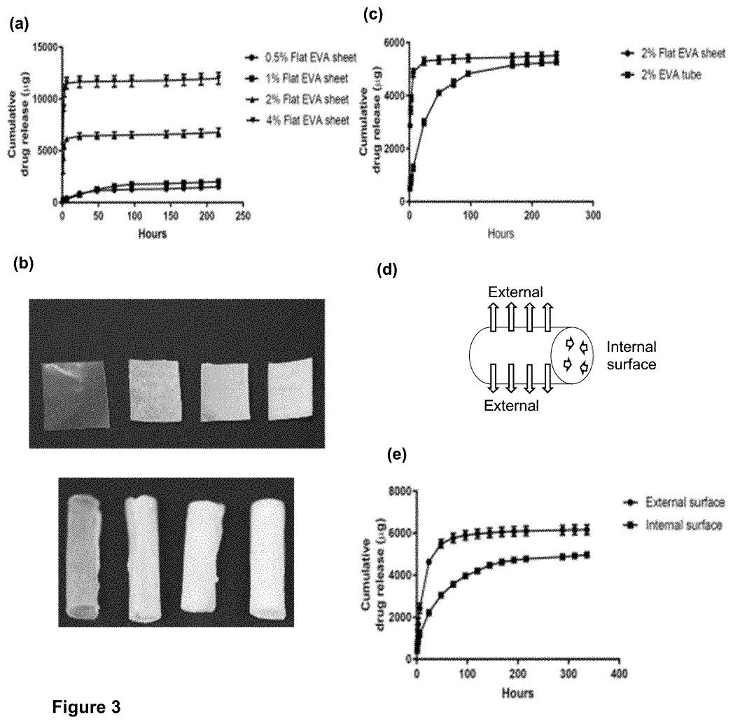

In vitro drug release from ibuprofen-loaded EVA membranes and tubes was rapid in the first 4 h and then plateaued at 24 h. Over 30% of the drug was released in the first 4 h with a 4% drug load while only 12% was released with the 0.5% drug load over the same time. The 2% drug load achieved the highest proportional release over 10 days at 92%, whereas, for 0.5%, 1% and 4% drug loading, 84%, 65% and 67% drug release respectively were observed (FIG. 3(a)). Therefore, the 2% drug load concentration was taken forward for further investigation. Furthermore, under the conditions used for drug release experiments, it was found that a tube geometry slowed the release profile in the first few days compared to flat sheets, with the release plateauing at 5 days (FIG. 3(b)). Release from EVA tubes was explored further by blocking drug release from one surface (either internal or external) by coating with blank EVA, allowing drug release from the opposite surface to be isolated and measured. The external surface had a cumulative drug release of 6165 pg in 14 days in comparison to 4970 pg from the internal surface (FIG. 3(d)).

Polycaprolactone (PCL)

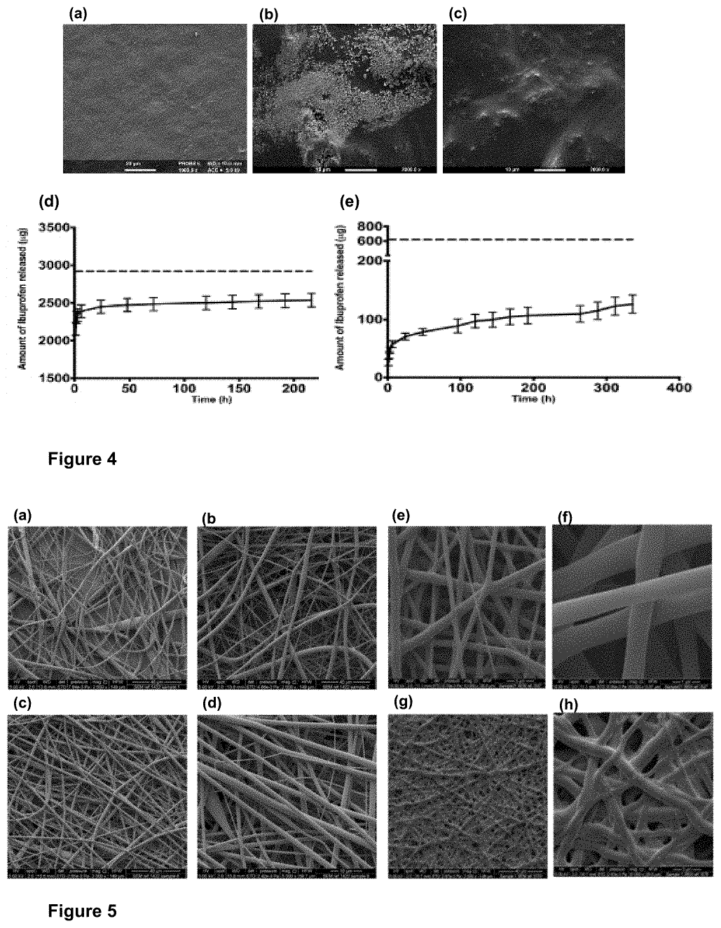

The composition and morphology of the PCL membranes either blank or loaded with 2% ibuprofen or ibuprofen-loaded MSN were analysed using SEM (FIG. 4). No pores could be seen and small crystals of drug were deposited over the surface of the membrane (FIG. 4(b)). Drug loading was random and not homogenous, however, the homogeneity improved with MSN loading c.f pure drug (FIG. 4(c)) and less drug was deposited on the surface.

Drug Release

In vitro drug release from ibuprofen-loaded and MSN-loaded PCL membranes was rapid in the first 4 h and then plateaued and remained constant after 24 h. The release of ibuprofen was slower when loaded into MSN before embedding into PCL (FIG. 4(e)). After 14 days ˜80% of drug was released in the ibuprofen loaded PCL, whereas, only ˜20% of ibuprofen was released from the MSN-loaded PCL. Embedding the drug into MSN and PCL produced a dual release mechanism which improved the controlled release of ibuprofen.

Poly (Lactic-Co-Glycolic) Acid (PLGA)

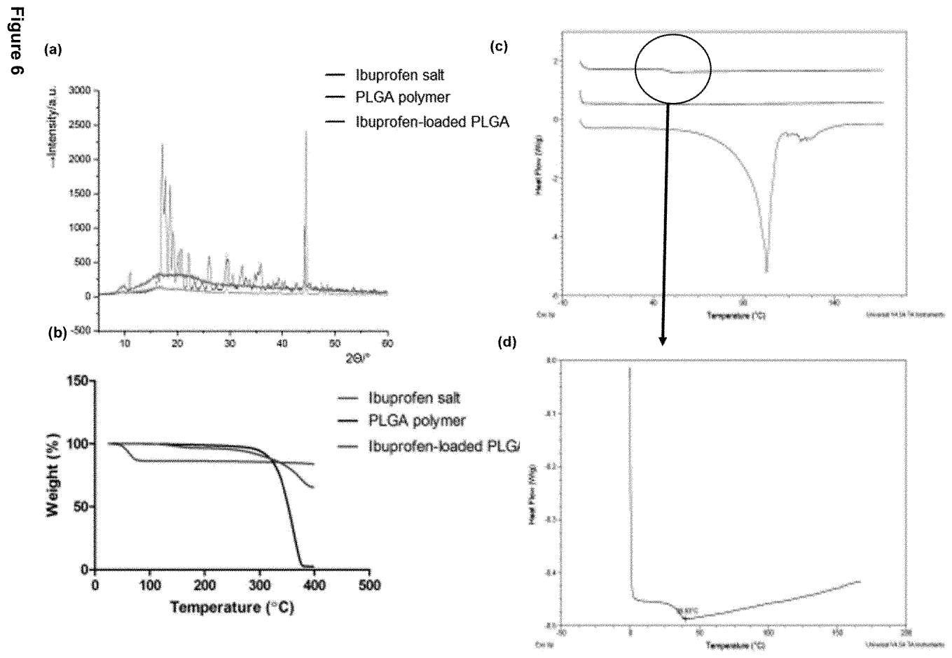

The composition and morphology of the electrospun PLGA nanofibres with and without ibuprofen or sulindac sulfide were analysed using SEM (FIG. 5). The nanofibres with a 1:7 ratio of drug to polymer were smooth and randomly aligned with an average diameter of 0.92 μm.

X-ray diffraction (XRD) analysis displayed a distinctive profile of the drug loaded material in comparison to the two starting materials (FIG. 6(a)). FIG. 6(b) Differential scanning calorimetry (DSC) demonstrated molecular dispersion of the drug as the glass transition peak of PLGA shifted to the lower temperature which may be due to plastcisation.

Drug Release

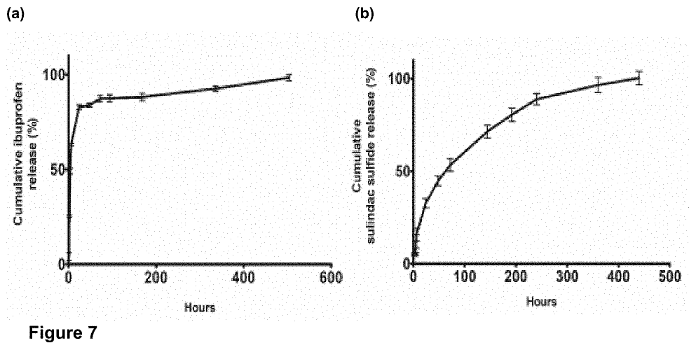

In vitro drug release from ibuprofen and sulindac sulfide-loaded PLGA nanofibres was measured every hour for 6 h, then every 24 h for 7 days and at 14 and 21 days. The ibuprofen-loaded PLGA nanofibres demonstrated a rapid release with ˜88% of the drug released within 7 days (FIG. 7(a)). The sulindac sulfide-loaded PLGA nanofibres demonstrated first order release, with 100% of the drug released within 20 days (FIG. 7(b)).

Effect of Drug-Loaded Materials on Nerve Regeneration

Having established various approaches for incorporating ibuprofen and sulindac sulfide into materials that might be useful in a nerve injury scenario, two selected formulations were taken forwards to test the concept in vivo using a rat model. The first selected formulation was ibuprofen-loaded EVA which provided a robust and reproducible method for testing the local delivery of ibuprofen to a nerve transection injury. EVA tubes were threaded onto transected nerves at the time of repair in order to test the concept of local release of ibuprofen from a biomaterial during the days following injury. The outcome measure of interest was a histological analysis of the number of neurites that had regrown across the transection site and entered the distal stump at 21 days. For completeness, functional outcome measures were also recorded along with histology to detect vascular changes in the nerve tissue.

A subsequent study then explored the delivery of ibuprofen and also sulindac sulfide using a PLGA nanofibrous wrap. The wrap option enabled the material to be deployed in a nerve crush model which isolated neuronal regeneration rate from other factors such as pathfinding that can influence recovery in more severe (transection) models. Because the nerve crush model is less severe than a transection, functional recovery is expected within 28 days. This allowed a range of functional outcome measures to be explored alongside histological analysis.

Ibuprofen-Loaded EVA

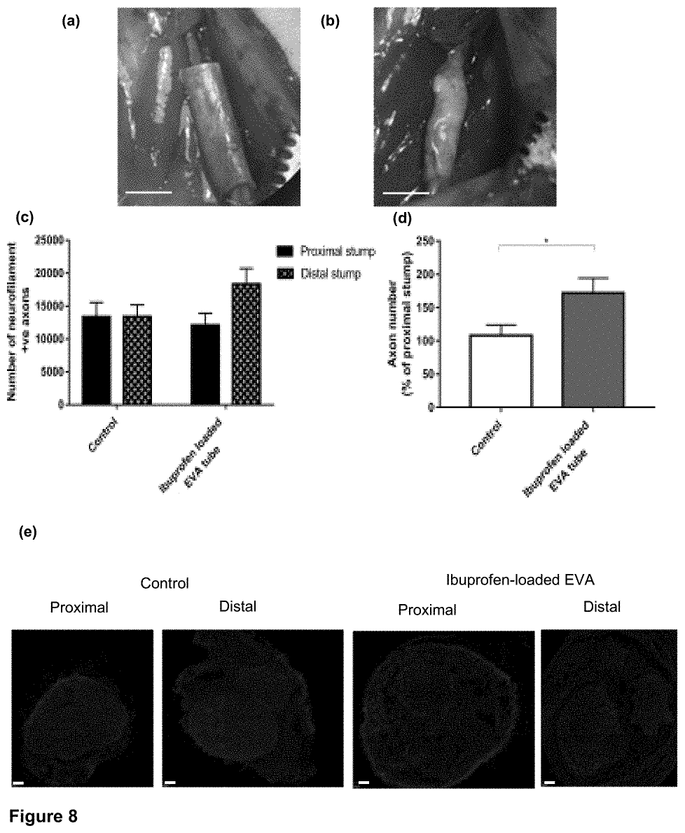

Immunodetection of neurofilament-positive neurons in transverse sections showed that EVA loaded with 2% ibuprofen increased the number of axons in the distal stump 21 days after transection injury in comparison to EVA with no drug (FIG. 8(c)). Furthermore, the number of axons as a percentage of the proximal stump was higher in the ibuprofen treatment group in comparison to the control group with 160% compared with 105% respectively (FIG. 8(d)).

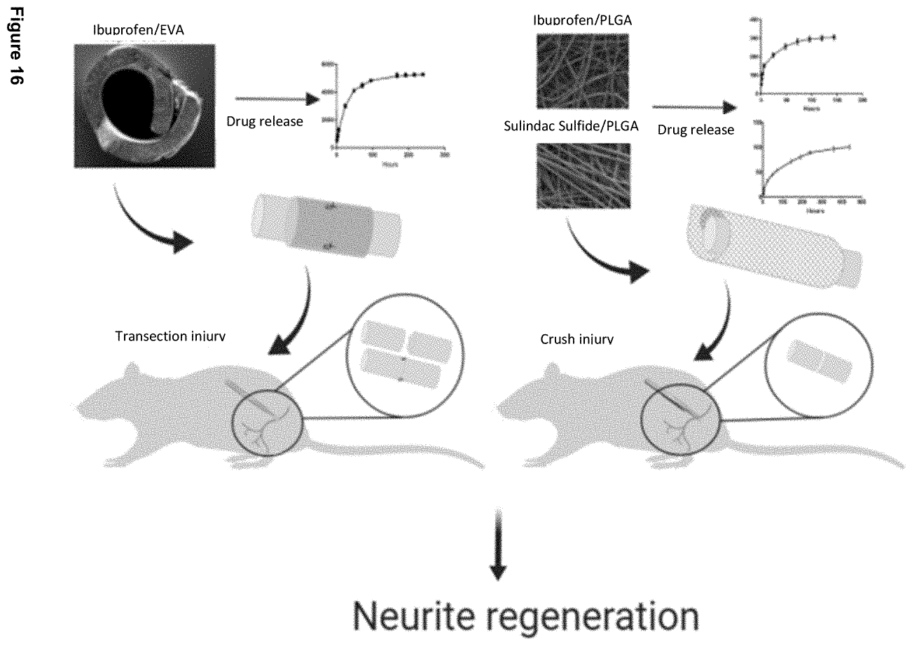

Motor and sensory recovery was studied at the 21 day end point using muscle weight, static sciatic index, electrophysiology, and von Frey analysis. Deficiencies were seen in the injured nerves compared to contralateral uninjured nerves with all measures and in all treatment/control groups, indicating that the transection model resulted in a loss of nerve function that persisted at the 21 day time point (FIG. 15).

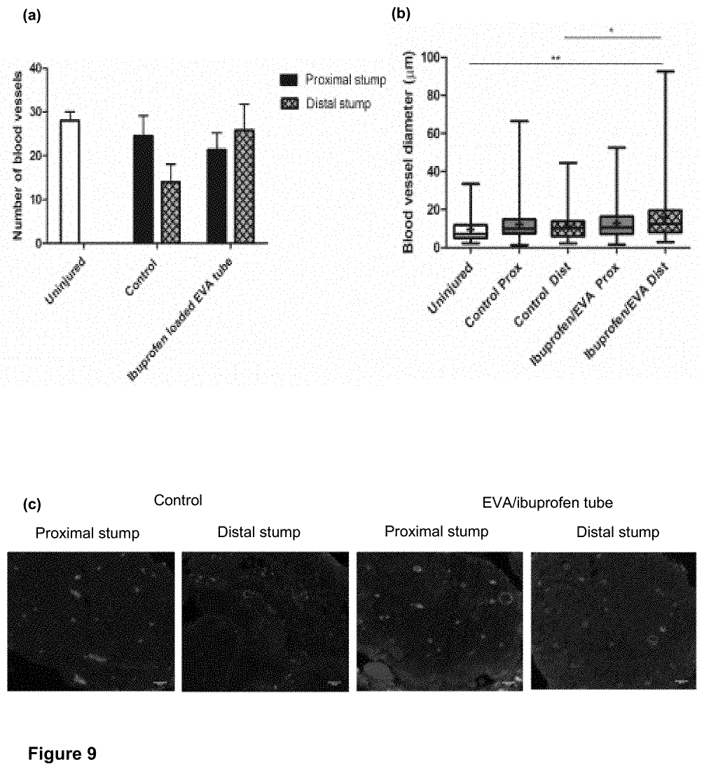

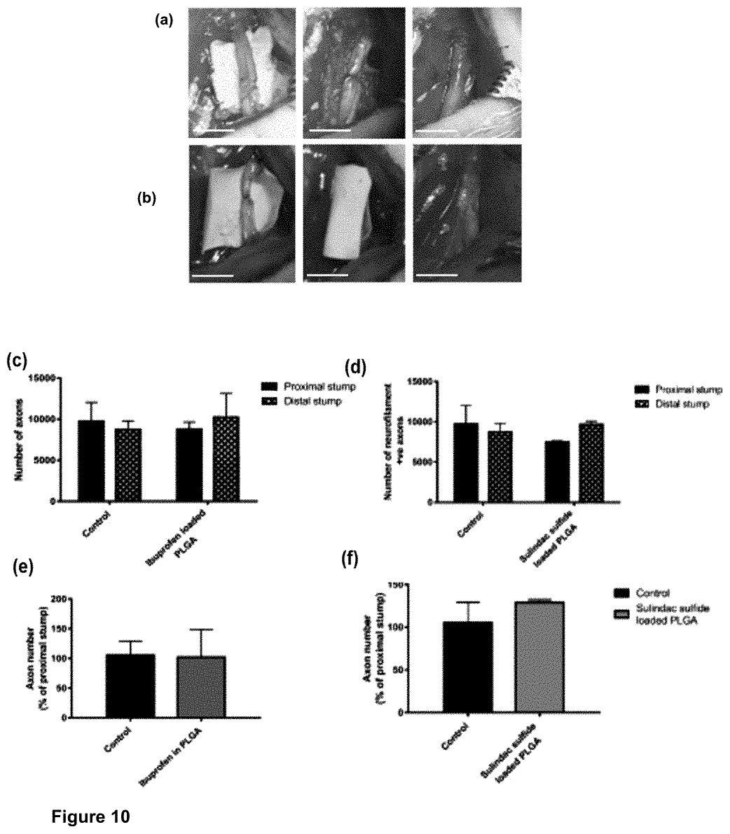

Vascularisation was examined via immunohistochemical staining of transverse sections for RECA-1. Analysis revealed the presence of blood vessels throughout the injured nerves in both the proximal and distal sections. A higher number and larger blood vessel diameter was observed in the distal stump of the ibuprofen treated group in comparison with the control group. Vasculature in the injured nerves in the ibuprofen-treated group revealed ˜25 blood vessels per nerve with a mean diameter of ˜18 μm, whereas, the control group presented ˜15 blood vessels per nerve with a diameter of ˜12 μm (FIG. 9).

Ibuprofen and Sulindac Sulfide-Loaded PLGA

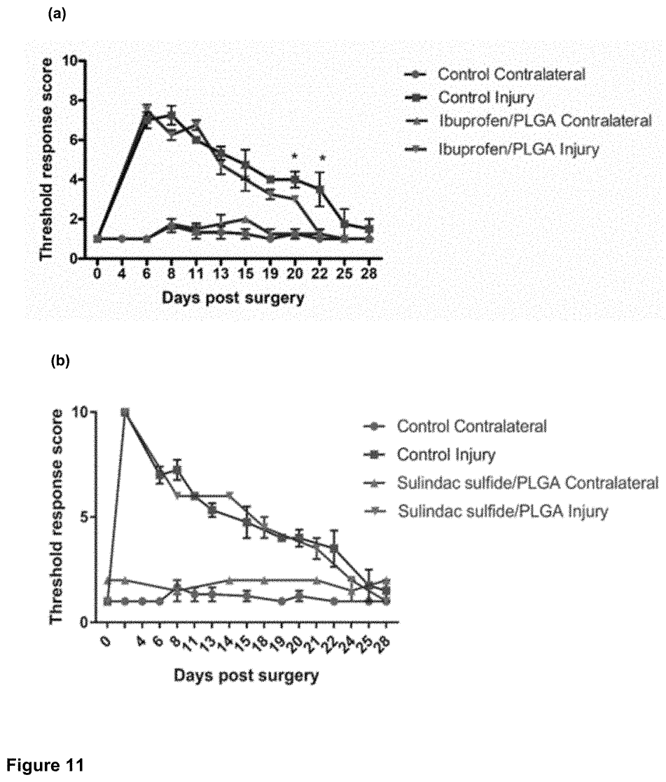

PLGA nanofibre sheets loaded with ibuprofen or sulindac sulfide were surgically implanted into a rat sciatic nerve as a wrap around a crush injury then assessed over 28 days. The PLGA nanofibres had appropriate handling properties for surgical implantation around the injured nerve (FIG. 11(a), (b)). Transverse sciatic nerve sections were stained to detect neurofilament immunoreactivity in order to quantify axon numbers. The results demonstrated that both ibuprofen and sulindac sulfide showed a trend towards increased numbers of axons in the distal stump in comparison to the control, however, the differences were not statistically significant (FIG. 11(c-f)).

Muscle mass and electrophysiology were assessed at the 28 day end point and SSI and von Frey were measured every 2-3 days throughout the experiment. No differences were seen in the threshold response to the von Frey filaments with sulindac sulfide treatment (FIG. 11(b)). However, the threshold response returned to baseline quicker with ibuprofen-loaded PLGA nanofibres with statistically significant improvement at 20 and 22 days post-injury in comparison with the control group (FIG. 11(a)).

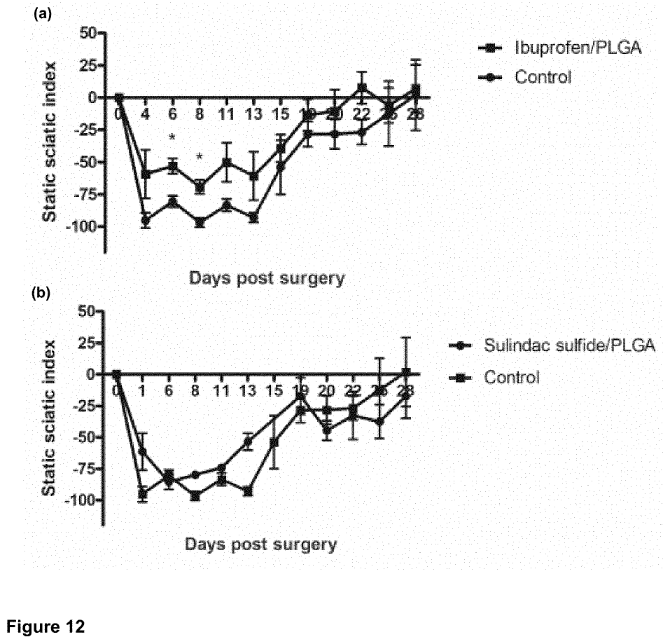

SSI was continuously lower in the ibuprofen treatment group from day 4 with statistical significance seen at days 6 and 8. The SSI also returned to baseline quicker in the ibuprofen treatment group than the control. This occurred by 22 days post-injury with ibuprofen-loaded PLGA nanofibers, but by day 28 in the control group (FIG. 12(a)). Small differences were seen in the sulindac sulfide treatment group in comparison to the control, but improvements between day 1 and 11 post injury can be observed. SSI also returned to baseline quicker in the treatment group that the control group, 25 and 28 days respectively (FIG. 12(b)).

No differences was seen with the gastrocnemius muscle mass with either ibuprofen or sulindac sulfide treatment (data not shown).

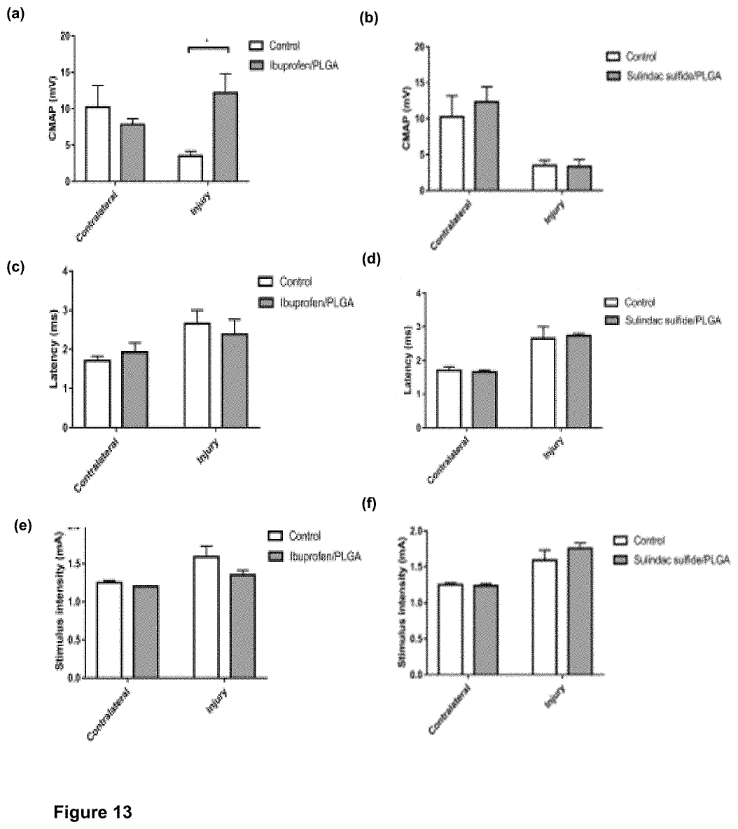

Electrophysiology was used to investigate the response of the gastrocnemius muscle to electrical stimulation of the proximal nerve. CMAPs were recorded from the gastrocnemius muscle in the contralateral and injured side in all animals. The CMAP in the ibuprofen treated group was significantly higher than that seen in control animals (FIG. 13(a)). There were small reductions in latency (FIG. 13(c)) and required stimulus intensity (FIG. 13(e)) in the ibuprofen treatment group in comparison with the control group although these were not statistically significant. No difference was observed in the CMAP, latency or the stimulus intensity between sulindac sulfide treatment group and the control (FIG. 13 (b, d, and f)).

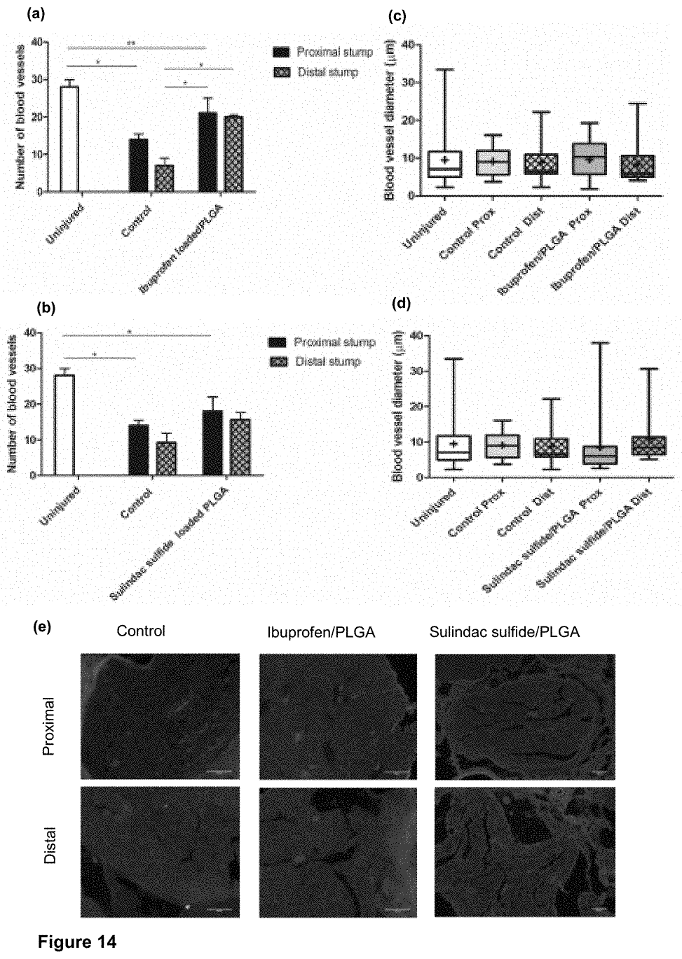

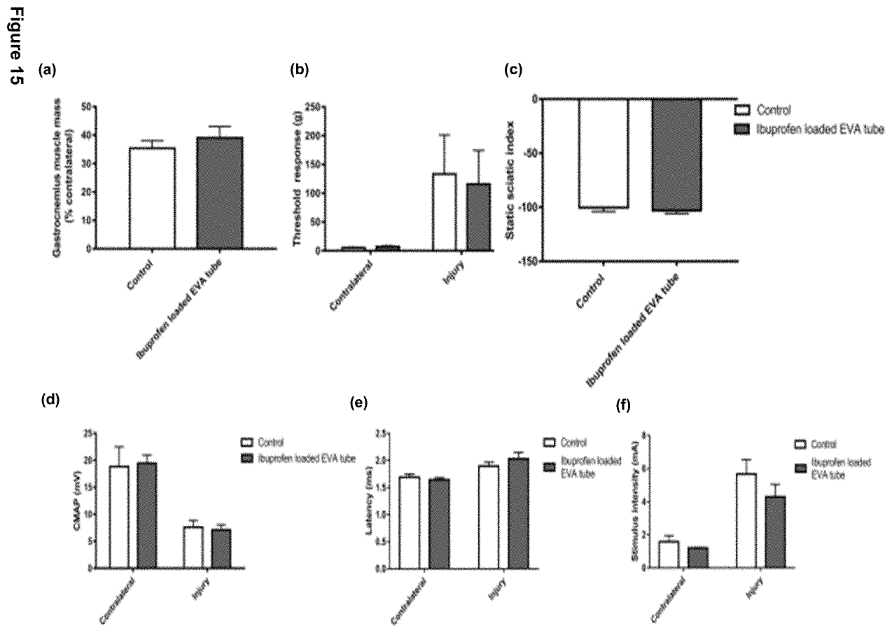

Vascularisation analysis demonstrated a higher number of blood vessels in the proximal stump in comparison with the distal stump with both drugs. There was an increase in blood vessel number observed with ibuprofen treatment at 28 days post-injury (FIG. 14(a)). A difference was observed between the two groups with more blood vessels present in the distal stump of the sulindac sulfide treated group in comparison with the control group but this was not statistically significant (FIG. 14(b)). Furthermore, larger blood vessel diameters were found in the sulindac sulfide treatment group (FIG. 14(d)).

### DISCUSSION

Drug loading into EVA provided drug release over 2 weeks. This was the initial target treatment duration, as a previous study had seen positive effects on regeneration following three weeks systemic ibuprofen treatment⁸. With the EVA membranes there was an initial burst release in the first 4 hours with 60% and 20% of drug released from the membranes and tubes respectively, then within 24 hours this subsided. This was consistent with a previous study that also observed a burst release of ibuprofen from EVA in the first few hours with 50% of the initial loaded drug released within the 24 hours, then the release subsided after 48 hours with the remainder of the drug released in 10 days²⁴. The EVA membranes could be successfully manufactured into tubes and it was evident that the geometry of the EVA affected the release rate in vitro with the tubes displaying a slower release. There is scope for future modifications of the tube design to maximise release from the lumen rather than the outer surface, thus increasing the proportion of administered drug released in the immediate proximity of the nerve tissue. Based on the in vitro data showing that EVA tubes could be used as a reproducible material for local delivery of ibuprofen these were tested in a rat sciatic nerve transection model. The primary outcome measure in this case was histological detection of the number of neurites that had grown into the distal stump and a 3 week period was chosen in order to allow time for the original neuronal structures to be cleared by Wallerian degeneration so that any changes in the number of new neurites crossing the transection site would be detected. Histological analysis of cross sections demonstrated an increase in axon number in the distal stump in the treatment group, which is consistent with previously reported data using osmotic pumps to deliver ibuprofen to transected nerves over 21 days⁹. Since the increase in the number of neurites detected in the distal stump of EVA-ibuprofen treated animals was greater than the number in the proximal stump, this indicates that ibuprofen may have been acting to increase sprouting as well as having an effect on accelerating neurite extension. A previous study that showed functional improvements with systemic ibuprofen treatment in a rat tibial nerve graft model reported similar numbers of axons in ibuprofen treatment and control groups distally at 12 weeks⁸. Assuming that local delivery of ibuprofen acts in a similar manner to systemic administration then this increased neurite number in the distal stump at 21 days would be expected to lead to a similar subsequent improvement in functional recovery, perhaps associated with increased maturation and myelination of axons at 12 weeks⁸.

While the action of ibuprofen on increased regeneration has been attributed to acceleration of neurite elongation as an agonist of PPARγ ^{8,9}, other mechanisms may contribute, for example the nerve vasculature which is associated with initial Schwann cell guidance and improved regeneration^{25 26}. Vascularisation was higher in the ibuprofen treatment group in terms of both number of blood vessels and blood vessel diameter observed here. Little is known about the mechanisms by which drugs modulate nerve regeneration via changes in vascularisation, so this observation is an important consideration and further investigation should explore whether it may be a cause or a consequence of increased neuronal growth.

EVA was a useful and well established biomaterial for initial testing of the hypothesis that local delivery of ibuprofen to nerves could be achieved, but while it has clinical applicability as a drug delivery material in other indications its non degradability makes it suboptimal for translation to clinical nerve repair applications. For this reason, additional studies were undertaken to develop approaches that could be used to deliver drugs to nerves using degradable materials more suitable for clinical translation.

Ibuprofen-loaded PCL demonstrated burst release of drug in the first four hours (22%), however, only 26% of drug had been released by day 10, which is typical behaviour of a biodegradable material²⁷. PCL is a commonly used biomaterial and has been used in PNI studies including attempts at drug delivery^{17,28} although the rapid initial release of ibuprofen from PCL membranes in this study precluded it being taken forward for in vivo testing. Embedding MSN loaded with ibuprofen²⁰ within PCL membranes improved the release profile, abrogating the initial burst of drug and providing a more controlled release which continued for 14 days. This provides a promising system to be explored further as a drug delivery platform for PNI.

Electrospinning PLGA with ibuprofen resulted in smooth, uniform and bead-free nanofibers with a diameter of ˜900 nm. The drug release from the PLGA exhibited first order kinetics, over 1 week, which is more sustained than results seen in a previous study where electrospun PLGA loaded with 10% ibuprofen exhibited a rapid release over the first 8 hours²⁹.

As PLGA is biodegradable (˜100 days to fully degrade when L:G ratio is 75:25 (Riggin et al., 2017)), and the electrospun PLGA formulation used here showed appropriate drug release properties it was taken forward for testing in vivo, using a crush model in which recovery of function could be monitored.

Histological analysis demonstrated an increase in axon number in the distal stump in the treatment group at 28 days when treated with both ibuprofen and sulindac sulfide. Interestingly, with ibuprofen the number of axons in the distal stump exceeded those in the proximal stump in the same animal, indicating increased sprouting as seen following treatment with ibuprofen-loaded EVA tubes.

There are no previous reports exploring the effect of sulindac sulfide for PNI, however, sulindac sulfide was previously shown to inhibit the activity of Rho in a concentration-dependent manner. The direct effect of sulindac sulfide on Rho activation was explored in SY5YAPP, HEK 293 and PC12 cells and levels of active Rho-GTP were reduced in all of the cell lines tested demonstrating that sulindac sulfide inhibits Rho activation³⁰. Therefore sulindac sulfide may have a similar effect to ibuprofen on increasing nerve regeneration.

The electrophysiological results, SSI and von Frey analysis all indicated that ibuprofen released from PLGA nanofibres improved functional recovery. These results further support the conclusion that local delivery of ibuprofen using a biomaterial can improve nerve regeneration following injury. Interestingly, PLGA-ibuprofen treatment had no effect on the number of blood vessels observed in the distal stump, but the blood vessel diameter was larger in the ibuprofen treatment group in comparison with the control group. Whereas, a higher number of blood vessels and a larger diameter was seen in the sulindac sulfide treatment group in comparison with the control group. Other NSAIDs and PPARγ agonists can be tested to determine whether they elicit similar beneficial effects as ibuprofen on nerve regeneration.

Further Testing on Drug Release, Degradation and Material Handling

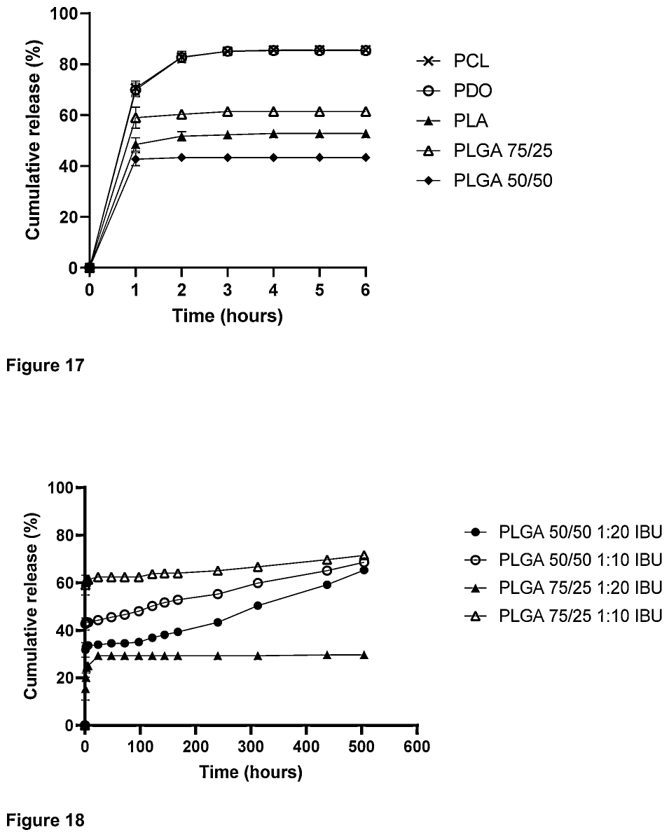

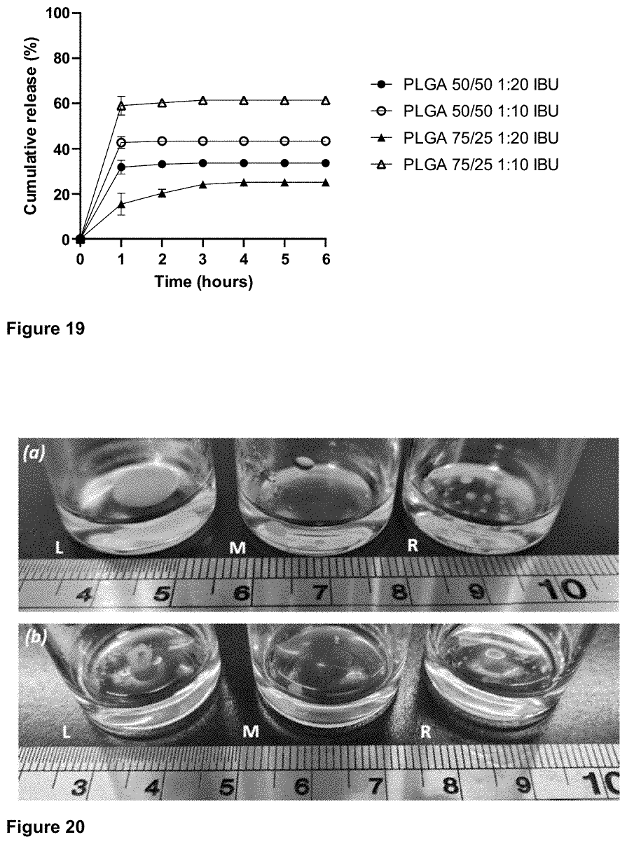

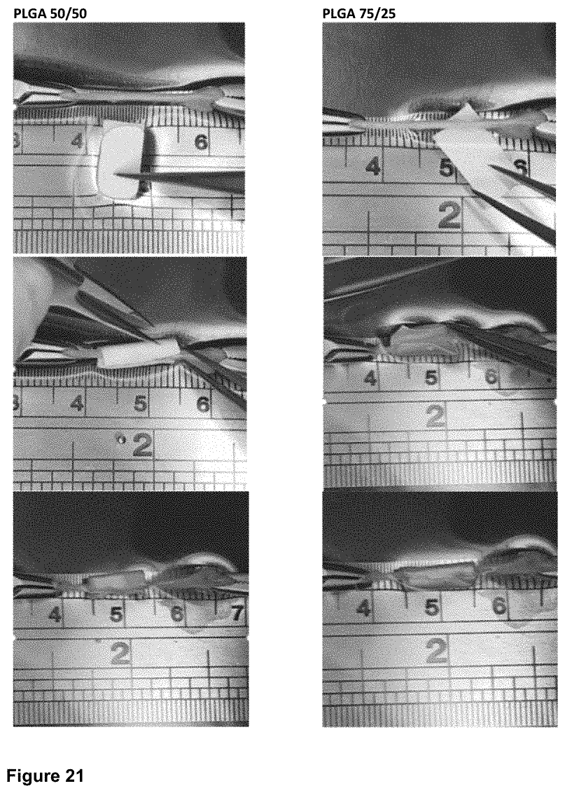

FIGS. 17 to 24 show the results of experiments testing drug (ibuprofen) release, degradation and material handling.

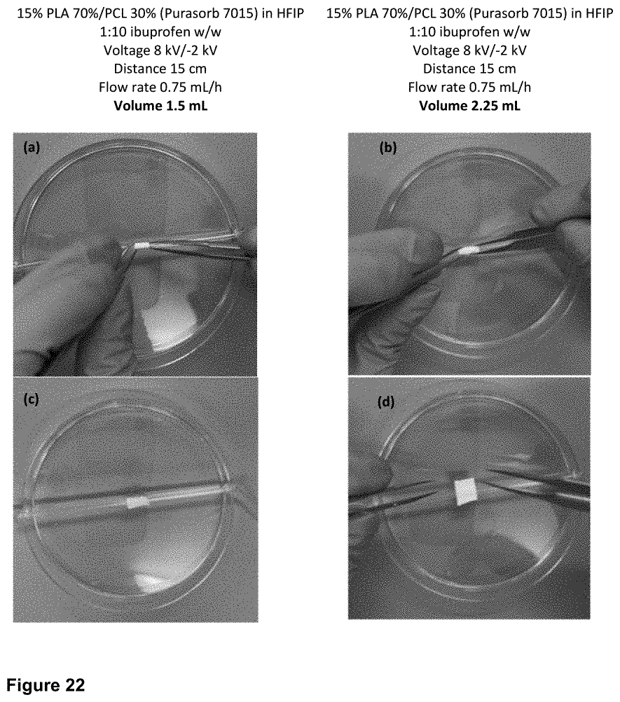

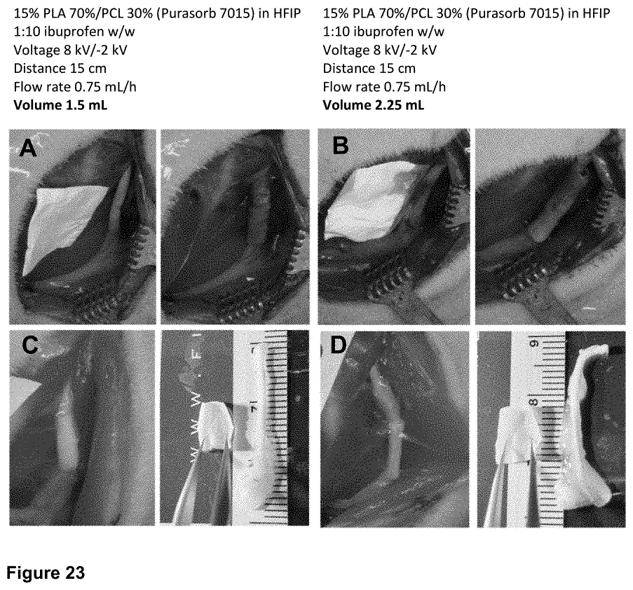

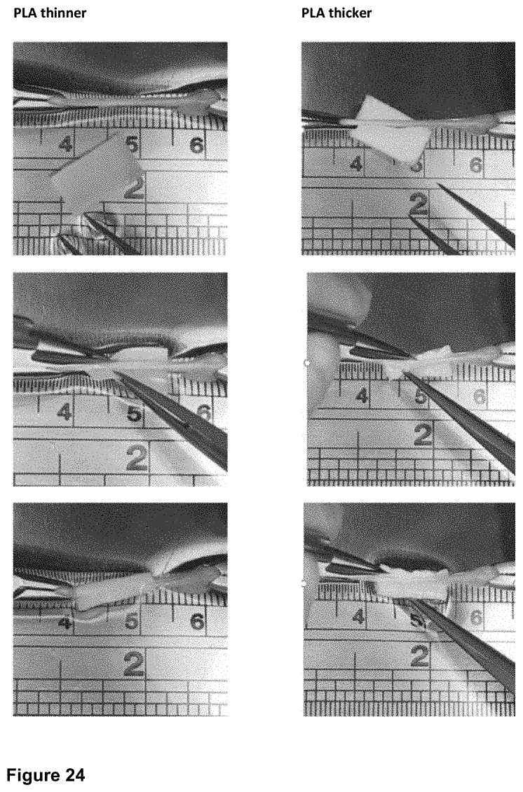

These data support a number of the concepts described above, in particular that drug release profiles can be tailored by changing the polymer type and composition, a constant rate of release can be achieved with some formulations (FIG. 18), the materials naturally degrade over time, and the handling properties of the materials can be tuned so they are suitable for use near a nerve (FIGS. 22-24). FIGS. 22 and 24 show respectively a silicone tube and an isolated nerve in vitro being used as a model to test handling properties, whereas FIG. 23 shows the materials being implanted into an in vivo (rat) model, where the original ‘before surgery’ dry material can be seen forming a wrap/patch around a nerve during surgery. Handling properties of materials of both thicknesses allowed successful implantation around the rat sciatic nerve (A, B). No fibrosis was observed after 21 days in vivo, and materials could easily be removed from the nerve (C,D). These ‘after surgery’ images show minimal fibrosis/adhesion/unwanted local tissue response.

The PLA/PCL materials in FIG. 23 have ideal handling properties. The thickness of these materials is thick enough to handle but thin enough to be able to wrap. The two materials in FIG. 23 were measured as approximately 75 and 125 micrometres thick.

FIG. 25 shows the same technology but with a different drug, dB-cAMP (dibutyryl cyclic adenosine monophosphate). This drug is not a PPAR gamma agonist but has been shown to have a positive effect on nerve regeneration. This material was made using PURASORB 5010, which is a PLGA 50/50 high molecular weight polymer (DL-lactide and Glycolide in a 50/50 molar ratio) and was selected for its theoretical degradation time of 3-4 months.

PLGA 50/50 provides a particularly useful release profile.

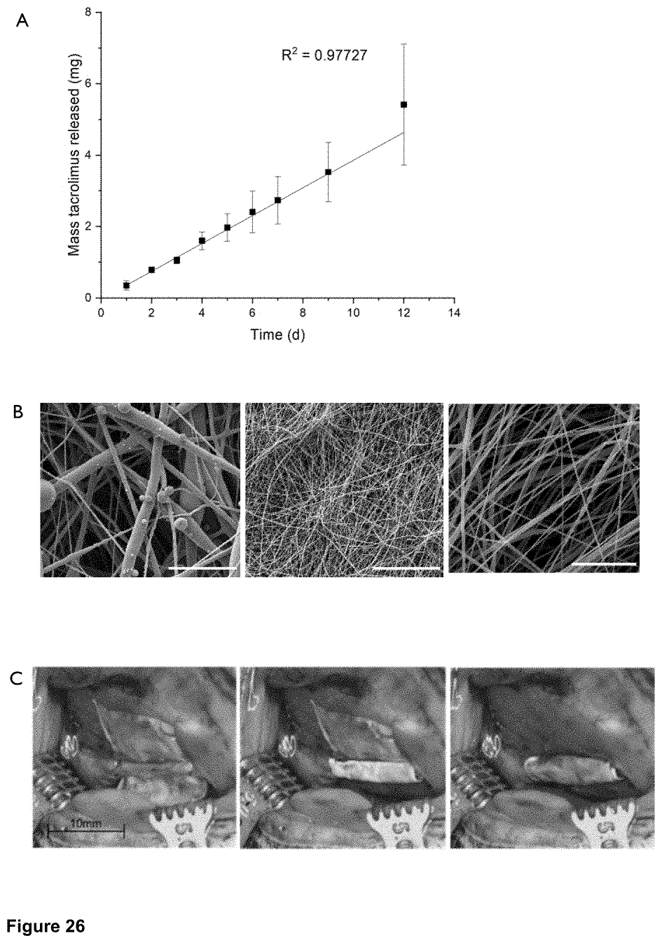

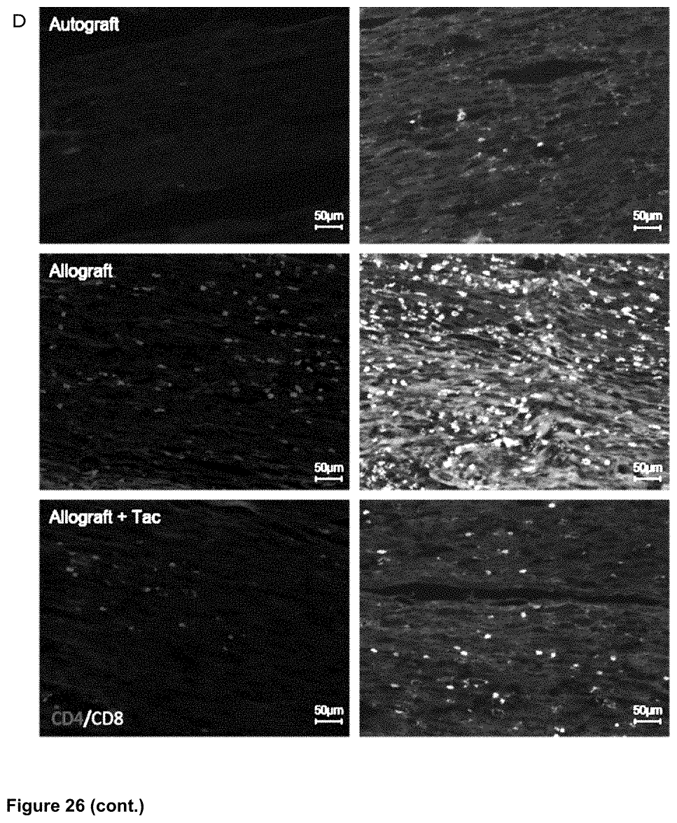

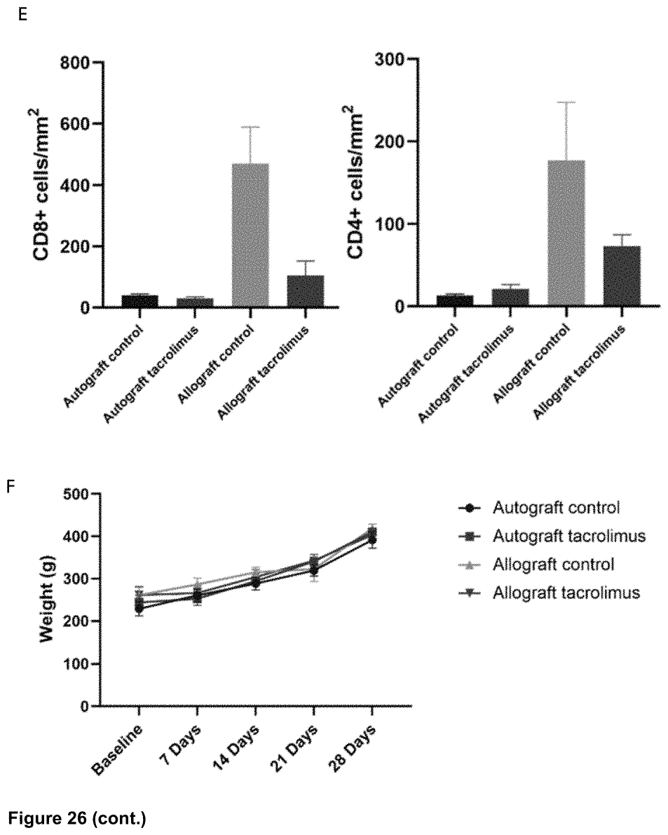

Data have also been generated (FIG. 26) showing that tacrolimus, a drug with pro-regenerative effects on nerves, can also be delivered locally using the biomaterials of the invention. Tacrolimus is also immunosuppressive, and can therefore be used to improve allograft acceptance and simultaneously accelerate regeneration. The data show that a beneficial structure and release profile is obtained using tacrolimus and a biomaterial of the invention, and also that it causes local immunosuppression in vivo.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

The invention described and claimed in this patent application relates in particular to a drug eluting nerve wrap or bandage, for treating a peripheral nerve injury (PNI).

The experiments described above prepare and characterise drug-loaded EVA, PCL and PLGA membranes and tubes, and demonstrate particular benefits for drug-embedded electrospun PLGA including favourable drug release kinetics. In vivo data in transection and crush injury peripheral nerve models are provided using EVA and PLGA membranes loaded with a PPARγ agonist (ibuprofen or sulindac sulphide) wrapped around the injured peripheral nerve, demonstrating a number of favourable therapeutically-relevant properties including increased neurites, blood vessel formation and regenerated nerve function. Data are also provided for materials comprising dB-cAMP or tacrolimus.

A particular embodiment of the invention relates to the local delivery of a PPARγ agonist such as ibuprofen using a PLGA membrane wrapped around an injured peripheral nerve, to improve neurite growth and functional recovery following a PNI. In addition to eluting the drug, the material of the invention can in some embodiments provide further advantages by acting as a physical support when wrapped around the damaged peripheral nerve, in the manner of a bandage, to aid recovery. This can be used for both crush injuries and transection injuries.

Wrapping an injured peripheral nerve in the material of the invention is advantageous. Furthermore, a PPARγ agonist such as ibuprofen is shown to be surprisingly effective at regenerating the nerve when administered locally. The data presented herein demonstrate the effective treatment of PNI using EVA and PLGA. PLGA is biodegradable and showed favourable release properties, and a PLGA wrap loaded with ibuprofen is shown to enhance nerve regeneration.

### REFERENCES

1. Deumens R, Bozkurt A, Meek M F, et al. Repairing injured peripheral nerves: Bridging the gap. Prog Neurobiol 2010: 92: 245-76.
2. Hoke A, Brushart T. Introduction to special issue: Challenges and opportunities for regeneration in the peripheral nervous system. Exp Neurol 2010: 223: 1-4.
3. Federici T, Liu J K, Teng C L Yang J, Boulis N M. A means for targeting therapeutics to peripheral nervous system neurons with axonal damage. Neurosurgery 2007: 60: 911-8; discussion 11-8.
4. Kuffler D P. An assessment of current techniques for inducing axon regeneration and neurological recovery following peripheral nerve trauma. Prog Neurobiol 2014: 116: 1-12.
5. Bhangra K S, Busuttil F, Phillips J B, Rahim A A. Using Stem Cells to Grow Artificial Tissue for Peripheral

Nerve Repair. Stem Cells Int 2016: 2016: 7502178.
6. Busuttil F, Rahim A A, Phillips J B. Combining Gene and Stem Cell Therapy for Peripheral Nerve Tissue Engineering. Stem Cells Dev 2017: 26: 231-38.
7. Chan K M, Gordon T, Zochodne D W, Power H A. Improving peripheral nerve regeneration: from molecular mechanisms to potential therapeutic targets. Exp Neurol 2014: 261: 826-35.
8. Madura T, Tomita K, Terenghi G. Ibuprofen improves functional outcome after axotomy and immediate repair in the peripheral nervous system. J Plast Reconstr Aesthet Surg 2011: 64: 1641-6.
9. Rayner M, Laranjeira S, Evans R, et al. Developing an in vitro model to screen drugs for nerve regeneration. Anat Rec (Hoboken), 2018. 301(10): p. 1628-1637′.
10. Fu Y, Kao W J. Drug release kinetics and transport mechanisms of non-degradable and degradable polymeric delivery systems. Expert Opin Drug Deliv 2010: 7: 429-44.
11. Schneider C, Langer R, Loveday D, Hair D. Applications of ethylene vinyl acetate copolymers (EVA) in drug delivery systems. J Control Release 2017: 262: 284-95.
12. Zhang W, Gao Y, Zhou Y, et al. Localized and sustained delivery of erythropoietin from PLGA microspheres promotes functional recovery and nerve regeneration in peripheral nerve injury. Biomed

Res Int 2015: 2015: 478103.
13. Zhang B. Potential use of ethylene vinyl acetate copolymer excipient in oral controlled release application: A literature review. Celanese 2015: 1-10.
14. Neal R A, Tholpady S S, Foley P L, et al. Alignment and composition of laminin-polycaprolactone nanofiber blends enhance peripheral nerve regeneration. J Biomed Mater Res A 2012: 100: 406-23.
15. Sun M, Kingham P J, Reid A J, et al. In vitro and in vivo testing of novel ultrathin PCL and PCL/PLA blend films as peripheral nerve conduit. J Biomed Mater Res A 2010: 93: 1470-81.
16. ClinicalTrials.gov. A Phase I Trial of a Novel Synthetic Polymer Nerve Conduit ‘Polynerve’ in Participants With Sensory Digital Nerve Injury (UMANC). 2016.
17. Salmoria G V, Paggi P A, Castro F, et al. Development of PCL/Ibuprofen tube for pripheral nerve regeneration. 2016.
18. Wang Z, Han N, Wang J, et al. Improved peripheral nerve regeneration with sustained release nerve growth factor microspheres in small gap tubulization. Am 0.1Transl Res 2014: 6: 413-21.
19. Giret S, Wong Chi Man M, Carcel C. Mesoporous-Silica-Functionalized Nanoparticles for Drug Delivery. Chemistry 2015: 21: 13850-65.
20. Zhang H, Li Z, Xu P, et al. Synthesis of novel mesoporous silica nanoparticles for loading and release of ibuprofen. J Control Release 2011: 152 Suppl 1: e38-9.
21. Benito C, Davis C M, Gomez-Sanchez J A, et al. STAT3 Controls the Long-Term Survival and Phenotype of Repair Schwann Cells during Nerve Regeneration. J Neurosci 2017: 37: 4255-69.
22. Bervar M. Video analysis of standing—an alternative footprint analysis to assess functional loss following injury to the rat sciatic nerve. J Neurosci Methods 2000: 102: 109-16.
23. Schneider C A, Rasband W S, Eliceiri K W. NIH Image to ImageJ: 25 years of image analysis. Nat

Methods 2012: 9: 671-5.
24. Thai Q A, Oshiro E M, Tamargo R J. Inhibition of experimental vasospasm in rats with the periadventitial administration of ibuprofen using controlled-release polymers. Stroke 1999: 30: 140-7.
25. Cattin A L, Burden A, Van Emmenis L, et al. Macrophage-Induced Blood Vessels Guide Schwann Cell-Mediated Regeneration of Peripheral Nerves. Cell 2015: 162: 1127-39.
26. Best T J, Mackinnon S E. Peripheral nerve revascularization: a current literature review. J Reconstr

Microsurg 1994: 10: 193-204.
27. Yang W W, Pierstorff E. Reservoir-based polymer drug delivery systems. J Lab Autom 2012: 17: 50-8.
28. Allen C, Eisenberg A, Mrsic J, Maysinger D. PCL-b-PEO micelles as a delivery vehicle for FK506: assessment of a functional recovery of crushed peripheral nerve. Drug Deliv 2000: 7: 139-45.
29. Canton I, McKean R, Charnley M, et al. Development of an Ibuprofen-releasing biodegradable PLA/PGA electrospun scaffold for tissue regeneration. Biotechnol Bioeng 2010: 105: 396-408.
30. Zhou Y, Su Y, Li B, et al. Nonsteroidal anti-inflammatory drugs can lower amyloidogenic Abeta42 by inhibiting Rho. Science 2003: 302: 1215-7.

1. A nanofibrous material comprising a drug, wherein the nanofibrous material comprising the drug is for treating a peripheral nerve injury by delivering the drug locally to a damaged or injured nerve.

2. The nanofibrous material comprising a drug according to claim 1, wherein the nanofibrous material is able to be wrapped around a peripheral nerve.

3. The nanofibrous material comprising a drug according to claim 1, wherein the nanofibrous material is biodegradable.

4. The nanofibrous material comprising a drug according to claim 1, wherein the nanofibrous material comprises polylactic-co-glycolic acid (PLGA) a PolyLactic Acid-Poly(ε-Caprolactone) copolymer, or polycaprolactone (PCL).

5. The nanofibrous material comprising a drug according to claim 1, wherein the nanofibrous material is formed by electrospinning.

6. The nanofibrous material comprising a drug according to claim 1, wherein the nanofibrous material is in the form of a sheet, membrane, bandage, wrap or patch.

7. The nanofibrous material comprising a drug according to claim 1, wherein:
(a) the nanofibrous material has a stiffness when in contact with the damaged or injured nerve, that is similar to or less than the stiffness of the nerve;
(b) the nanofibrous material has a thickness between about 10 micrometres and about 1000 micrometres; or
(c) a combination of (a) and (b).

8. The nanofibrous material comprising a drug according to claim 1, wherein the drug is embedded or encapsulated within the nanofibrous material.

9. The nanofibrous material comprising a drug according to claim 1, wherein the drug is complexed with a nanoparticle or microparticle.

10. The nanofibrous material comprising a drug according to claim 1, wherein the drug is a Non Steroidal Anti Inflammatory Drug (NSAID) or a peroxisome proliferator-activated receptor (PPAR) agonist.

11. The nanofibrous material comprising a drug according to claim 1, wherein the drug is ibuprofen, dB-cAMP, or tacrolimus.

12. The nanofibrous material comprising a drug according to claim 1, wherein the nanofibrous material delivers a sustained-release of the drug to the nerve.

13. A drug delivery system for delivering a drug locally to a nerve, wherein the drug delivery system comprises the nanofibrous material comprising a drug as defined in claim 1.

14. (canceled)

15. (canceled)

16. (canceled)

17. A method of treating a peripheral nerve injury in a patient in need thereof, comprising contacting a damaged nerve with the nanofibrous material comprising a drug according to claim 1.

18. The method according to claim 17, wherein the method comprises wrapping the damaged nerve in the nanofibrous material.

19. The method according to claim 17, wherein the peripheral nerve injury is a crush, a partial transection, a complete transection, a gap, or caused by a neuropathy.

20. (canceled)

21. A method of manufacturing a nanofibrous material comprising a drug, wherein the drug is incorporated into the nanofibrous material at an amount suitable to effect sustained release of an efficacious dose when in contact with a nerve in vivo.

22. A kit for preparing a nanofibrous material comprising a drug, the kit comprising:
a coaxially electrospun nanofibrous PLGA sheet and ibuprofen, and instructions for combining those components; or
a drug and polymer solutions for electrospinning into a nanofibrous PLGA sheet comprising the drug, and instructions for electrospinning the sheet.

23. The kit according to claim 22, wherein the sheet has a size and dimension suitable to wrap around a peripheral nerve and wherein the ibuprofen is at a therapeutically-effective dose.

24. (canceled)

25. (canceled)

26. The nanofibrous material according to claim 4, wherein the polymer is PLGA 50/50, wherein lactide and glycolide monomers are present in a 50/50 molar ratio.

27. The nanofibrous material of claim 5, wherein the nanofibrous material is an electrospun PLGA comprising peroxisome proliferator-activated receptor gamma (PPARγ) agonist.
